(19) United States
(12) Patent Application Publication
(10) Pub. No.: US 2025/0121118 A1
(43) Pub. Date: Apr. 17, 2025

(54) SYSTEMS, APPARATUSES, KITS, AND METHODS FOR MODIFICATION OF TISSUE USING A PHOTO-INITIATOR AND LIGHT

(71) Applicant: DurVena Inc., Overland Park, KS (US)

(72) Inventors: Fernando Buarque, Overland Park, KS (US); John Ashley, Overland Park, KS (US); Michael McCormick, Overland Park, KS (US)

(21) Appl. No.: 18/913,461

(22) Filed: Oct. 11, 2024

Related U.S. Application Data
(60) Provisional application No. 63/544,051, filed on Oct. 13, 2023.

(51) Int. Cl. A61L 27/36; A61F 2/06

(52) U.S. Cl. CPC A61L 27/3687; A61F 2/06; A61L 27/3625; A61F 2240/001; A61L 2430/34; A61L 2430/40

(57) ABSTRACT

Disclosed herein are systems, methods, kits, and apparatuses for tissue modification. The systems can include a light chamber and a vein carrier. The light chamber can have one or more light source assemblies to provide light to a vein in the vein carrier.

### CROSS-REFERENCE TO RELATED APPLICATIONS

This application claims the benefit of U.S. application Ser. No. 63/544,051, filed Oct. 13, 2023, the entire contents of which are incorporated herein by reference in their entirety.

### FIELD OF DISCLOSURE

The present disclosure relates to systems, methods, kits, and apparatuses for modification of tissue using a photo-initiator and light. The system can include a light chamber and a vein carrier. The light chamber can have one or more light source assemblies to provide light to a vein in a vein carrier.

### BACKGROUND

Modification of tissue can provide many benefits in medical applications. Modified tissues can improve by-pass anastomosis by strengthening veins for implantation into a patient's body. Modification of tissue using photo-initiators and light can preserve normal tissue architecture, reduce post-surgical inflammation, and reduce or prevent development of pathogenic collagen bundles and adhesions following surgical procedures. Further, tissue modification can be used to prevent intimal hyperplasia within a human blood vessel. Tissue modification can also be used to reduce reactivity of tissue surfaces to prevent adhesions and/or contracture. However, tissue modification systems and methods known in the art fail to provide sterile environments for tissue modification. Furthermore, tissue modification systems and methods known in the art fail to efficiently treat tissues without causing damage to the tissue.

Therefore, there is a need for efficient and sterile systems and methods for tissue modification.

### SUMMARY

The present disclosure generally relates to a system for tissue modification of a vein. The system can include a vein carrier and a light chamber. The vein carrier can include a translucent holder and a vein attachment mechanism disposed within the translucent holder. The light chamber can be configured to receive the vein carrier. The light chamber can include one or more light source assemblies configured to provide light to the translucent holder of the vein carrier.

In some aspects, the light chamber can include a fixed light tower and a moveable light tower. In some aspects, the fixed light tower and the moveable light tower can be operable to surround the vein carrier. In some aspects, the translucent holder can include a fixed translucent section and a moveable translucent section. In some aspects, the light chamber can further include a docking bay having a connection mechanism operable to receive a connector of the vein carrier. In some aspects, the translucent holder is operable to maintain sterility of the vein. In some aspects, each of the one or more light assemblies can include at least one light source and at least one lens configured to direct light to the vein through the translucent holder.

In some aspects, the vein carrier can further include a vein carrier temperature control assembly operable to cool and/or humidify the vein within the translucent holder. In some aspects, the vein carrier temperature control assembly can include at least one fan, at least one vent, a fluid chamber, and one or more humidifiers. In some aspects, the light chamber can include a light chamber temperature control assembly operable to cool the light chamber. In some aspects, the vein attachment mechanism can have an adjustable length. In some aspects, the system can further include a controller operable to control one or more parameters of the system. In some aspects, the one or more parameters can include temperature, humidity, light intensity, light wavelength, and/or light spectrum.

Further provided herein is a system for tissue modification. The system can include a light chamber and a vein carrier. The light chamber can include a fixed light tower, a moveable light tower, and a light chamber temperature control assembly. The fixed light tower can include one or more light assemblies. The moveable light tower can include one or more light assemblies. The vein carrier can include a translucent holder operable to secure the vein. The translucent holder can include a fixed translucent section, a moveable translucent section, and a vein attachment mechanism operable to secure the vein within the translucent holder. The vein carrier can include a vein carrier temperature control assembly operable to control a temperature and/or humidity within the translucent holder. The translucent holder can be configured to maintain sterility of the vein.

Further provided herein is a method for modifying a vein. The method can include obtaining the vein from a patient, coating the vein with a photo-initiator, securing the vein within a translucent holder of a vein carrier, receiving the vein carrier in a light chamber, and providing a light, via the light chamber, to the vein through the translucent holder, thereby activating the photo-initiator and modifying the vein. The translucent holder can be operable to maintain sterility of the vein.

In some aspects, the light can include a wavelength of about 530 nm to about 540 nm. In some aspects, the photo-initiator can include one or more of xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, Rose Bengal (“RB”), erythrosine, riboflavin, methylene blue (MB), Toluidine Blue, Methyl Red, Janus Green B, Rhodamine B base, Nile Blue A, Nile Red, Celestine Blue, Remazol Brilliant Blue R, riboflavin-5-phosphate (R-5-P), N-hydroxypyri-dine-2-(I H)-thione (N-HTP), or photoactive derivatives thereof. In some aspects, the light can be provided by two or more light sources. The two or more light sources can provide light sequentially. In some aspects, the method can further include implanting the vein into the patient. In some aspects, the method can further include maintaining a temperature and/or humidity within the translucent holder via a vein carrier temperature control assembly.

### BRIEF DESCRIPTION OF FIGURES

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1 is a perspective view of a system for tissue modification in one aspect.

FIG. 2 is a perspective view of a system for tissue modification in one aspect.

FIG. 3 is a perspective view of a system for tissue modification in one aspect.

FIG. 4 is a perspective view of a light chamber in one aspect.

FIG. 5 is a perspective view of a light chamber in one aspect.

FIG. 6A is a perspective view of a light chamber in one aspect.

FIG. 6B is a partial cross sectional top view of a light chamber in one aspect.

FIG. 7 is a perspective view of a vein carrier in one aspect.

FIG. 8 is a perspective view of a vein carrier in one aspect.

FIG. 9A is a partial perspective view of a vein carrier in one aspect.

FIG. 9B is a partial perspective view of a vein carrier in one aspect.

FIG. 10A is a top view of a vein carrier in one aspect.

FIG. 10B is a partial cross sectional view of a vein carrier in one aspect.

FIG. 11 is a flowchart illustrating a method in one aspect.

FIG. 12 is a perspective view of a vein carrier in one aspect.

FIG. 13A is a partial perspective view of a vein carrier in one aspect.

FIG. 13B is a partial perspective view of a vein carrier in one aspect.

FIG. 14A is a perspective view of a component of a vein carrier in one aspect.

FIG. 14B is a perspective view of a component of a vein carrier in one aspect.

FIG. 15 is a schematic diagram of a controller in one aspect.

FIG. 16 illustrates the system in one aspect.

FIG. 17A illustrates an uncoated vein.

FIG. 17B illustrates a coated vein.

FIG. 18 is a graph illustrating maximum force before separation for treated veins and untreated veins.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment,” “an embodiment,” or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, “vein,” “tissue,” “artery,” and “vessel” can be used to describe various biological components for use with the systems and methods described herein. It will be appreciated that any biological component can be used with the systems and methods described herein.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Provided herein are systems, apparatuses, kits, and methods for modification of tissue using a photo-initiator and light. The systems, apparatuses, kits, and methods can be used to strengthen tissue (e.g., vein tissue) for use in medical procedures. The systems, apparatuses, kits, and methods described herein are operable to modify tissue while maintaining tissue sterility.

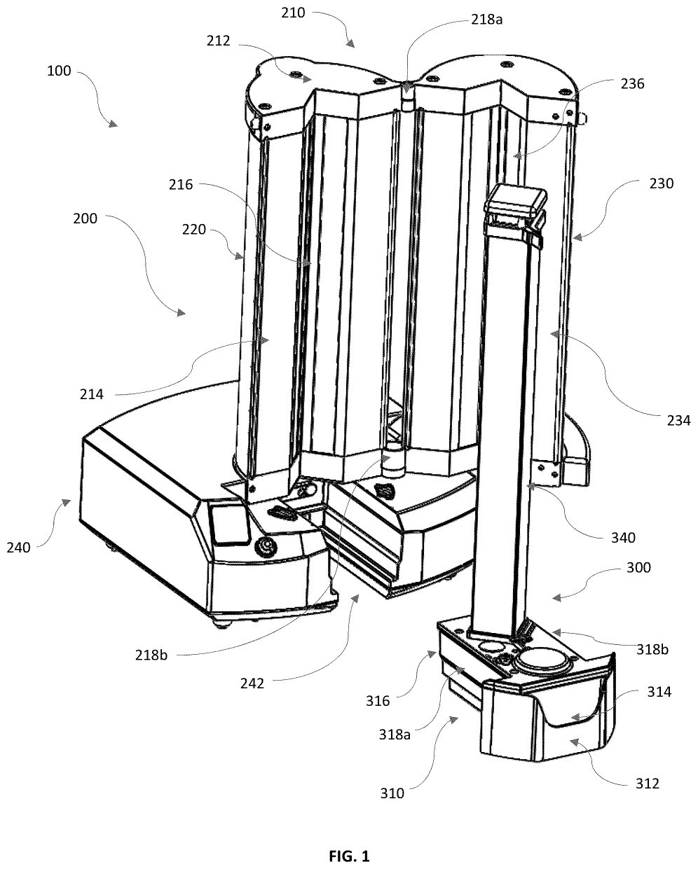

As illustrated in FIG. 1, the tissue modification system 100 can include a light chamber 200 (i.e., light apparatus) and a vein carrier 300 (i.e., vein carrier apparatus). The light chamber 200 can be an electronic instrument that can be plugged into line power or be battery powered. The vein carrier 300 is also an electronic instrument that can be battery powered, connected for power electronically to the light chamber 200, or plugged into line power.

The light chamber 200 can be operable to receive the vein carrier 300. In some aspects, the light chamber 200 can be operable to enclose the vein carrier 300. The vein carrier 300 can be operable to hold a vein. In some aspects, the vein carrier 300 can include a translucent material. In some aspects, the light chamber 200 can be operable to provide light to the vein through the translucent material of the vein carrier 300. The light chamber 200 can be operable to provide light from one or more directions via one or more light sources. In some aspects, the light chamber 200 can be configured to provide light which substantially covers the vein in the vein carrier 300. In some aspects, the light chamber 200 can further include one or more optical elements to ensure that substantially all of the vein is illuminated by the one or more light sources.

The light chamber 200 can include a support base and a delivery stand 210. The delivery stand 210 can include one or more light-emitting assemblies (e.g., one or more light sources and one or more optical elements). As illustrated in FIGS. 1-5, the delivery stand 210 can have a fixed light tower 220 and a moveable light tower 230. The fixed light tower 220 can be integral to the support base 240. The moveable light tower 230 can be moved relative to the support base 240 and the fixed light tower 220. The fixed light tower 220 and the moveable light tower 230 can be connected via one or more hinges. As illustrated in FIG. 1, the fixed light tower 220 and the moveable light tower 230 can be connected via an upper hinge 218(a) and a lower hinge 218(b). The connection of the fixed light tower 220 and the moveable light tower 230 can be described as a clamshell configuration. In another aspect, the fixed light tower 220 and the moveable light tower 230 can be connected with one or more screws or other common fasteners. In an aspect, the moveable light tower 230 can be fixed to the support base 240 and the support base 240 can have a sliding or rotating mechanism to move the moveable light tower 230. In other aspects, the delivery stand 210 can include only a fixed light tower 220. When the delivery stand 210 includes only a fixed light tower 220, the delivery stand 210 can receive the vein carrier 300 through an opening at an upper or lower end of the delivery stand 210.

In some aspects, the fixed light tower 220 and the moveable light tower 230 can be substantially the same size (e.g., each make up roughly 50% of the volume of the delivery stand 210). In other aspects, the fixed light lower 220 can be larger or smaller than the moveable light tower 230. The fixed light tower 220 can be about 10% to about 20%, about 20% to about 30%, about 30% to about 40%, about 40% to about 50%, about 50% to about 60%, about 60% to about 70%, about 70% to about 80%, about 80% to about 90%, or more of the volume of the delivery stand 210. The moveable light tower 230 can be about 10% to about 20%, about 20% to about 30%, about 30% to about 40%, about 40% to about 50%, about 50% to about 60%, about 60% to about 70%, about 70% to about 80%, about 80% to about 90%, or more of the volume of the delivery stand 210.

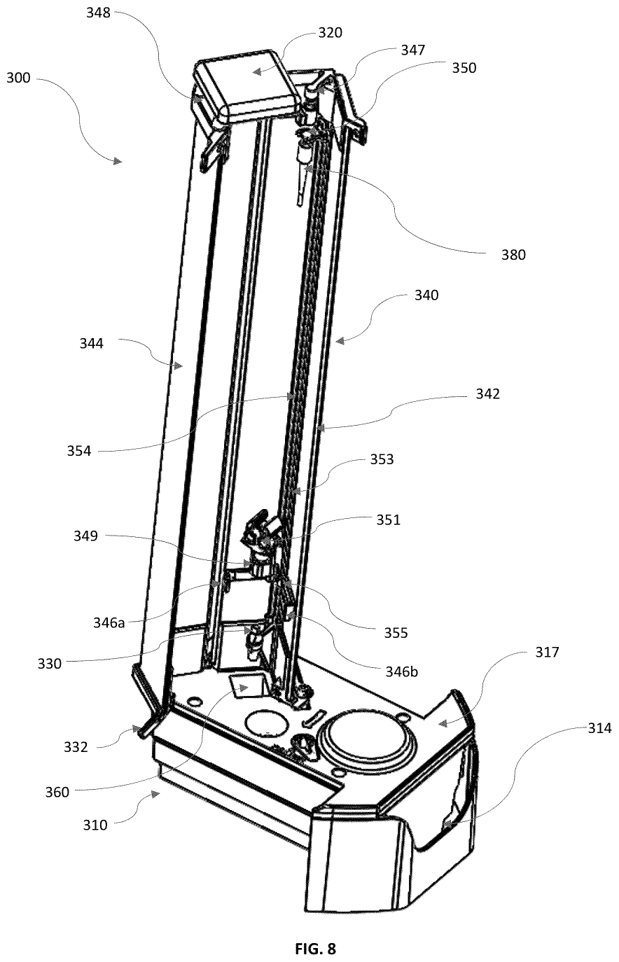

FIG. 8 illustrates a vein carrier 300. In an aspect, the vein carrier 300 can include a translucent holder 340. The translucent holder 340 can be operable to allow light to enter the vein carrier 300. In some aspects, the vein carrier 300 can include a lower support 310. The lower support 310 can include a front section 312 and a rear section 316. The front section 312 can include a handle 314. The handle 314 is configured to allow the vein carrier to be positioned relative to the light chamber 200. The rear section 316 can have two or more sides. As illustrated in FIG. 1, the rear section 316 of the vein carrier 300 can have sides 318(a), 318(b). The two or more sides 318(a), 318(b) can be configured to slide into and/or couple with the light chamber 200.

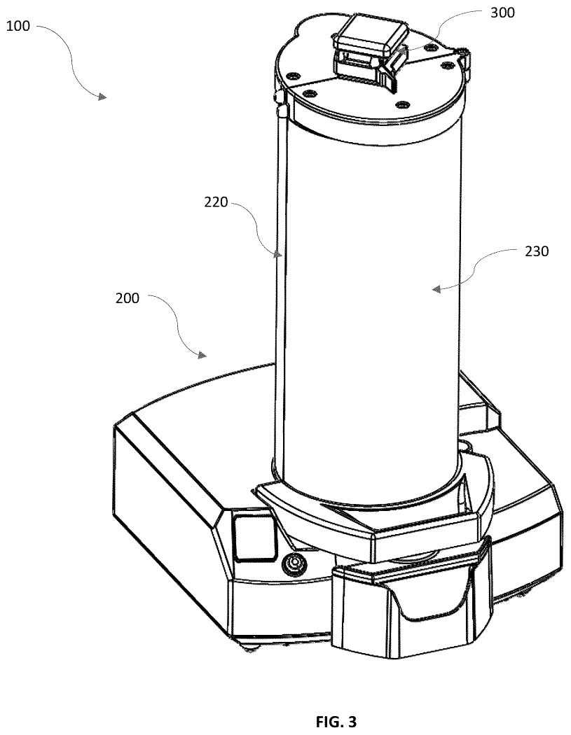

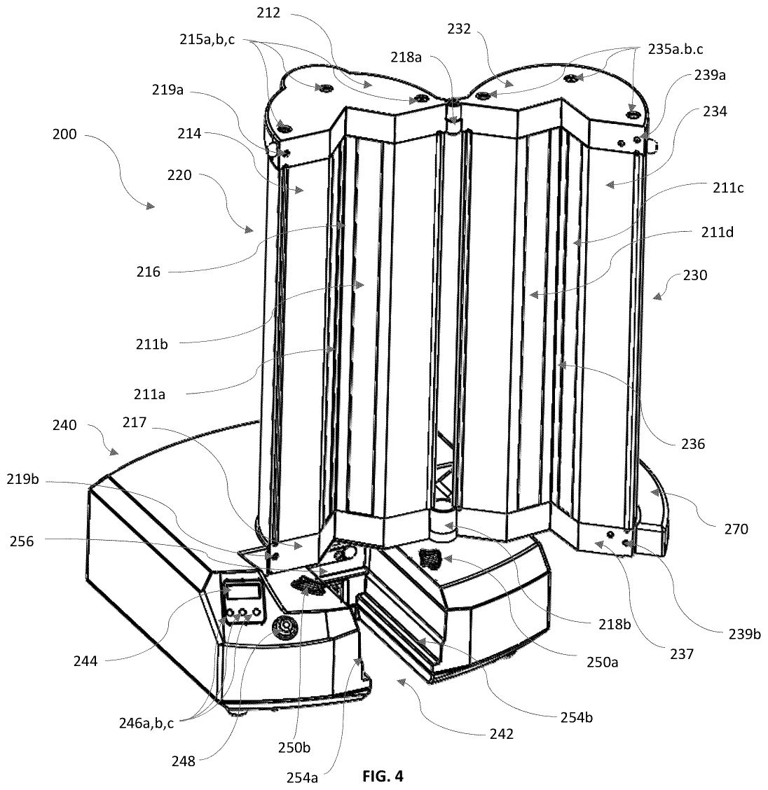

As illustrated in FIG. 4, the fixed light tower 220 can include an interior face 214 and a recess 216. The moveable light tower 230 can include an interior face 234 and a recess 236. The interior face 214 of the fixed light tower 220 is configured to seat next to the interior face 234 of the moveable light tower 230 when the delivery stand 210 is in the closed position, as illustrated, for example, in FIG. 3. The recess 216 of the fixed light tower 220 and the recess 236 of the moveable light tower 230 are configured to surround the translucent holder 340 of the vein carrier 300, as illustrated, for example, in FIG. 3.

As illustrated in FIG. 4, the support base 240 can include a docking bay 242 that is configured to receive the sides 318(a), 318(b) of the rear section 316 of the vein carrier 300. The docking bay 242 can be operable to hold the vein carrier 300 in the light chamber 200. As illustrated in FIG. 4, the docking bay 242 can include one or more rails and/or ledges 254(a), 254(b) on the sides of the docking bay 242. The one or more rails and/or ledges 254(a), 254(b) can be configured to mate with opposing rails and/or opposing ledges on the vein carrier 300 lower support 310. In some examples, other mating configurations can be used to mate the vein carrier 300 with the light chamber 200.

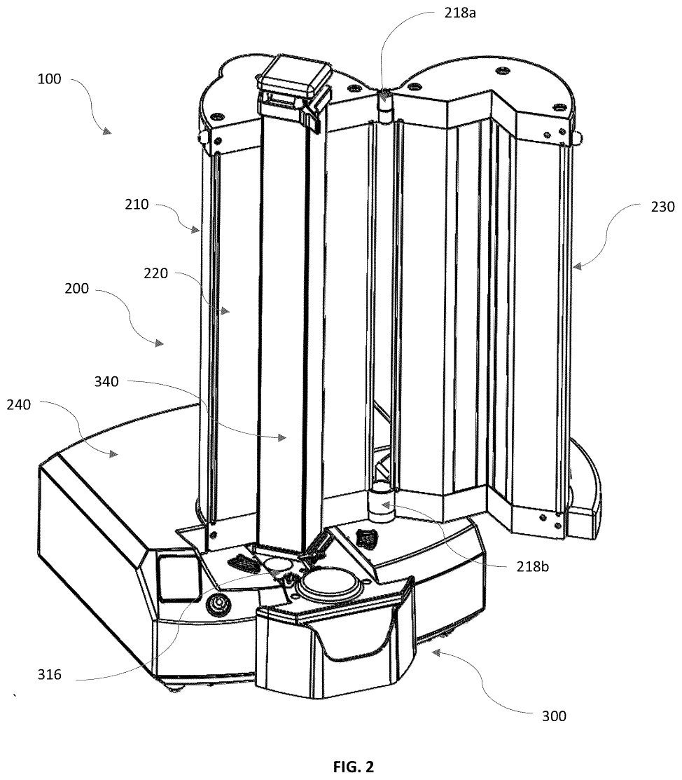

FIG. 2 illustrates the vein carrier 300 docked in the docking bay 242 of the light chamber 200. The rear section 316 of the vein carrier 300 is inserted into the docking bay 242 of the light chamber. The upper translucent holder 340 is adjacent the recess 216 of the fixed light tower 220. FIG. 2 illustrates the moveable light tower 230 in an open configuration (e.g., the moveable light tower 230 is not surrounding the vein carrier 300).

FIG. 3 illustrates the moveable light tower 230 in a closed configuration (e.g., the movable light tower 230 is surrounding the vein carrier 300). In the closed configuration, the interior face 214 of the fixed light tower 220 and the interior face 234 of the moveable light tower 230 are positioned adjacent to one another. The fixed light tower 220 and the moveable light tower 230 enclose the translucent holder 340 of the vein carrier 300.

As illustrated in FIG. 4, the fixed light tower 220 can have a top cap 212 and the moveable light tower 230 can have a top cap 232. The top caps 212, 232 provide structural support to the fixed light tower 220 and the moveable light tower 230, respectively. In an aspect, the fixed light tower 220 can be removably coupled to the moveable light tower 230 via one or more attachment mechanisms. The top cap 212 of the fixed light tower 220 can include a magnetic material 219(a) and a portion of hinge 218(a). The top cap 232 of the moveable light tower 230 can include a magnetic material 239(a) and a portion of hinge 218(a). The top caps 212, 232 can be connected via the hinge 218(a). In some aspects, the top caps 212, 232 can be removably coupled to the fixed light tower 220 and moveable light tower 230, respectively. When the top caps 212, 232 are uncoupled from the fixed light tower 220 and moveable light tower 230, the interior components of the fixed light tower 220 and moveable light tower 230 can be accessed (e.g., for maintenance, etc.).

In some aspects, the tissue modification system 100 can include one or more vents operable to allow air, and thereby heat, to flow out of the tissue modification system. The fixed light tower 220 and/or moveable light tower 230 can include one or more vents. The one or more vents can be operable to allow air, and thereby heat, to flow out of the tissue modification system 100. In some aspects, the top cap 212 of the fixed light tower 220 can include one or more vents. In an aspect, the top cap 212 of the fixed light tower 220 can include three vents 215(a), 215(b), 215(c). In some aspects, the top cap 232 of the moveable light tower 230 can include one or more vents. In an aspect, the top cap 232 of the moveable light tower 230 can include three vents 235(a), 235(b), 235(c). The one or more vents can be on located on an upper surface of the top caps 212, 232 as illustrated, for example, in FIG. 4. In other aspects, the one or more vents can be located on the sides of the top caps 212, 232. In yet another aspect, the one or more vents can be a gap between the top caps 212, 232 and the remaining portions of the fixed light tower 220 and the moveable light tower 230. The one or more vents can provide air flow to cool the inside of the fixed light tower 220 and the moveable light tower 230. In some aspects, air can be provided by a fan, pressurized air source, or other air supply devices located on the caps 212, 232 or connected to a tubing connected to the one or more vents. In an aspect, the tissue modification system 100 can have one or more fans configured to draw air out of the light chamber 200 through the one or more vents 215(a), 215(b), 215(c), 235(a), 235(b), 235(c). In some aspects, the one or more fans can be disposed within top cap 212 and/or top cap 232.

As illustrated in FIG. 4, the fixed light tower 220 can have a bottom base 217 (e.g., a bottom cap). The moveable light tower 230 can have a bottom base 237 (e.g., a bottom cap). The bottom bases 217, 237 can provide structural support to the fixed light tower 220 and the moveable light tower 230, respectively. The bottom base 217 of the fixed light tower 220 can connect the fixed light tower 220 to the support base 240. The bottom base 237 of the moveable light tower 230 connects the moveable light tower 230 to the support base. In some aspects, the bottom bases 217, 237 can be integral to the support base 240. In other examples, the bottom bases 217, 237 can be operable to connect to a connection port in the support base 240 such that the bottom bases 217, 237 are removable from the support base 240.

The bottom base 217 of the fixed light tower 220 can include one or more attachment mechanism. In some aspects, the one or more attachment mechanisms can include a magnetic material 219(b). In some aspects, a portion of hinge 218(b) can be coupled (e.g., connected) to the fixed light tower 220. For example, the portion of hinge 218(b) can be connected to the bottom base 217 of the fixed light tower 220. The bottom base 237 of the moveable light tower 230 can include one or more attachment mechanisms. In some aspects, the one or more attachment mechanisms can include a magnetic material 239(b). In some aspects, a portion of hinge 218(b) can be coupled (e.g., connected) to the moveable light tower 230. For example, the portion of hinge 218(b) can be connected to the bottom base 237.

The bottom bases 217, 237 can include one or more vents. The one or more vents on the bottom bases 217, 237 can be located on the sides or bottom exterior surfaces of the bottom bases 217, 237. In another aspect, the one or more vents on the bottom bases 217, 237 can be formed as gaps between the bottom bases 217, 237 and the remaining portions of the fixed light tower 220 and the moveable light tower 230, respectively. The one or more vents can be configured to provide a cooling air flow to the light chamber 200 and/or remove warm air (e.g., produced by the one or more light sources) from the light chamber 200. Air can flow from a fan in the support base 240 through the openings 250(a), 250(b) into the one or more vents in the bottom bases 217, 237 through the fixed light tower 220 and moveable light tower 230 and out the one or more vents 215(a), 215(b), 215(c), 235(a), 235(b), 235(c) in the top caps 212, 232. In an aspect, air flow can be provided by a fan, pressured air source, or other air supply devices in the support base 240 to the vents in the bottom bases 217, 237. It will be appreciated that air can flow from the one or more vents in the bottom bases 217, 237 to the one of more vents in the top caps 212, 232 and/or from the one or more vents in the top caps 212, 232 to the one or more vents in the bottom bases 217, 237. The air flow can allow the fixed light tower 220 and the moveable light tower 230 to maintain a safe operating temperature. In some aspects, the fan can be operable to provide air to the light chamber 200 any time the light chamber is in the closed configuration (e.g., the moveable light tower 230 is removably coupled to the fixed light tower 220). In other aspects, the fan can operate for desired durations while the light chamber 200 is in the open or closed configuration. In an example, the light chamber 200 can have one or more temperature sensors in communication with a control system (e.g., controller, processor, microcontroller, microprocessor, computing device, etc.) and the indicator screen operable to determine and control a temperature in the light chamber 200.

As illustrated in FIG. 4, the bottom base 237 of the moveable light tower 230 can include a handle 270. The handle 270 can be grasped by a user to move the moveable light tower 230 between the open configuration (e.g., as illustrated in FIG. 2) and the closed configuration (e.g., as illustrated in FIG. 3).

The magnetic materials 219(a), 219(b) on the fixed light tower 220 can be magnets. The magnetic materials 239(a), 239(b) on the moveable light tower 230 can be metals (e.g., iron, steel, stainless steel, and the like) that are attracted to the magnets on the fixed light tower 220. Alternatively, the magnetic materials 239(a), 239(b) can be magnets and the magnetic materials 219(a), 219(b) can be metals (e.g., iron, steel, stainless steel, and the like) that are attracted to the magnetic materials 239(a), 239(b). In another aspect, the magnetic materials 219(a), 219(b) can be magnets and the magnetic materials 239(a), 239(b) can be magnets having an opposite polarity, such that the magnetic materials 219(a), 219(b) are attracted to the magnetic materials 239(a), 239(b). In these aspects, the magnetic materials 219(a), 219(b) and magnetic materials 239(a), 239(b) are configured to removably couple to each other. In other aspects, other attachment mechanisms can be used to removable couple the fixed light tower 220 to the moveable light tower 230. Other attachment mechanisms can include a screw and hole configuration, latches, snap-fit mechanisms, or other mechanical attachment mechanisms known in the art. In an aspect, the moveable light tower 230 and the fixed light tower 220 can be locked in place when in the closed configuration. The moveable light tower 230 and the fixed light tower 220 can have opposing locking mechanisms configured to lock when the light chamber 200 is in the closed configuration. In an aspect, the locking mechanism can be any kind of lock (e.g., latch lock or the like) that is actuated between a locked position and unlocked position by a control system in electronic communication with the lock or manually by an operator. The control system can actuate the lock to a locked position when a treatment commences (e.g., light provided by the fixed light tower 220 and the moveable light tower 230). The control system can actuate the lock to the unlocked position when the treatment is completed. In some aspects, the magnetic materials 219(a), 219(b), 239(a), 239(b) can have sensors (e.g., position sensors, contact sensors, etc.) operable to communicate with the control system that the fixed light tower 220 is properly coupled to the moveable light tower 230.

In some aspects, the tissue modification system 100 can include one or more light sources and one or more lenses. In some examples, the one or more lenses can be configured to focus light from the one or more light sources on a vein or other biological component to be modified. As illustrated in FIG. 4, the recess 216 of the fixed light tower 220 can include one or more lenses to focus light on the upper translucent holder 340 of the vein carrier. In an aspect, the recess 216 of the fixed light tower 220 can include two lenses 211(a), 211(b) operable to focus light on two sides of the upper translucent holder 340, as illustrated, for example, in FIG. 4. In another aspect, the recess 236 of the moveable light tower 230 can include one or more lenses operable to focus light on the upper translucent holder 340 of the vein carrier 300. As illustrated in FIG. 4, the recess 236 of the moveable light tower 230 can include two lenses 211(c), 211(d) operable to focus light on two sides of the upper translucent holder 340. The lenses 211(a), 211(b), 211(c), 211(d) can provide focused light to the entirety of the translucent vein holder 340.

As illustrated in FIG. 4, the support base 240 can include a user interface configured to operate the light chamber 200. For example, the user interface can be configured to allow the user to select various parameters of the tissue modification system 100 (e.g., temperature, humidity, light intensity, light frequency, etc.). The user interface can also be configured to provide alerts to the user regarding the operation of the tissue modification system 100. The user interface can include a start button 248, an indicator screen 244, and indicator LEDs 246(a), 246(b), 246(c). The start button 248 can be used to activate light delivery from the light chamber 200 (e.g., via one or more light sources of the fixed light tower 220 and the moveable light tower 230) to the biological component (e.g., contained within the upper translucent holder 340). The indicator screen 244 can be a display configured to provide parameters of the light delivery. For example, the indicator screen 244 can display the intensity, wavelength, and/or other parameters of the light delivery. The indicator screen 244 can also display the time duration of light delivery. The indicator screen 244 can also display the light chamber 200 operation state (e.g., delivering, not delivering, and/or error). In some aspects, the indicator screen 244 can display errors for the light chamber 200 such as the moveable light tower 230 being improperly positioned for light delivery, failure to provide a desired light, and/or other errors. The indicator screen 244 can display the vein carrier 300 operation state (e.g., docked, not docked, closed, not closed, and/or error). In some aspects, the indicator screen 244 can display errors for the vein carrier 300 such as the vein carrier is not in the proper docking position, the vein carrier is not properly closed, the vein is not properly secured, and/or other errors. The indicator screen 244 can be an LCD screen, an LED screen, or another similar device. The indicator screen 244 can further provide alpha-numeric characters, images, and/or videos to provide information to the user. For example, the indicator screen 244 can display image or video instructions for connecting the various components of the tissue modification system 100. The indicator screen 244 can display a percentage of tissue modification completed. The indicator screen 244 can further be configured to display any desired information of the tissue modification system to the user. The indicator screen can further display readings from the one or more sensors described herein.

The indicator LEDs 246(a), 246(b), 246(c) can be green, yellow, and red, respectively, to indicate “on” (green), “ready” (yellow), and “off” (red). The indicator LEDs 246(a), 246(b), 246(c) can also be multicolored LEDs that are used to communicate more information to the user than just three states. For example, each LED 246(a), 246(b), 246(c) can be configured to provide a status of the light chamber 200, the vein carrier 300, and/or the tissue modification system 100. In other aspects, the indicator LEDs 246(a), 246(b), 246(c) can be configured to provide other information to the user.

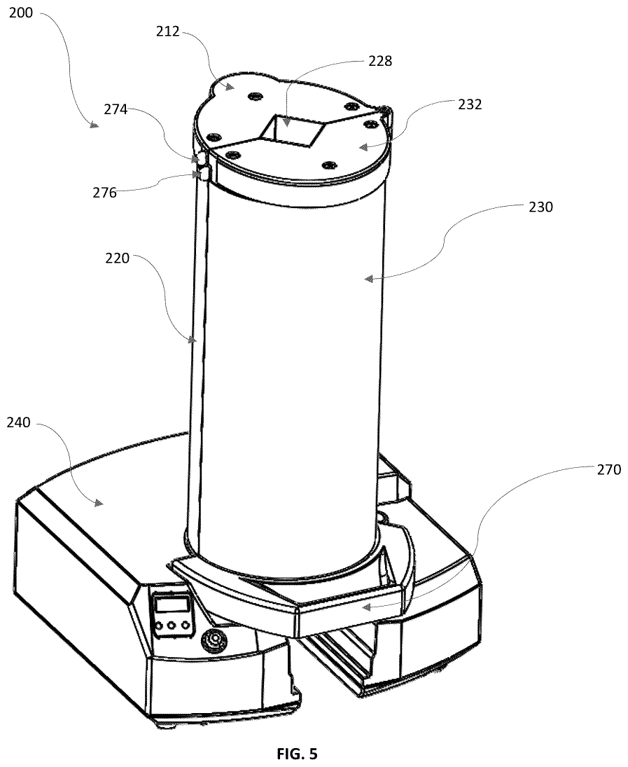

FIG. 5 illustrates the light chamber 200 in the closed configuration. The light chamber can include opening tabs 274, 276. Opening tab 274 can be a part of the top cap 212 of the fixed light tower 220. Opening tab 276 can be part of the top cap 232 of the moveable light tower 230. The opening tabs 274, 276 are configured to provide another mechanism for moving the moveable light tower 230 between the open configuration and the closed configuration. The opening tabs 274, 276 can be used in conjunction with, or alternatively to, the door handle 270. When the light chamber 200 is in the closed configuration, the top cap 212 of the fixed light tower 220 and the top cap 232 of the moveable light tower 230 can form a top opening 228 through which the top of the vein carrier 300 extends, as illustrated, for example, in FIG. 5.

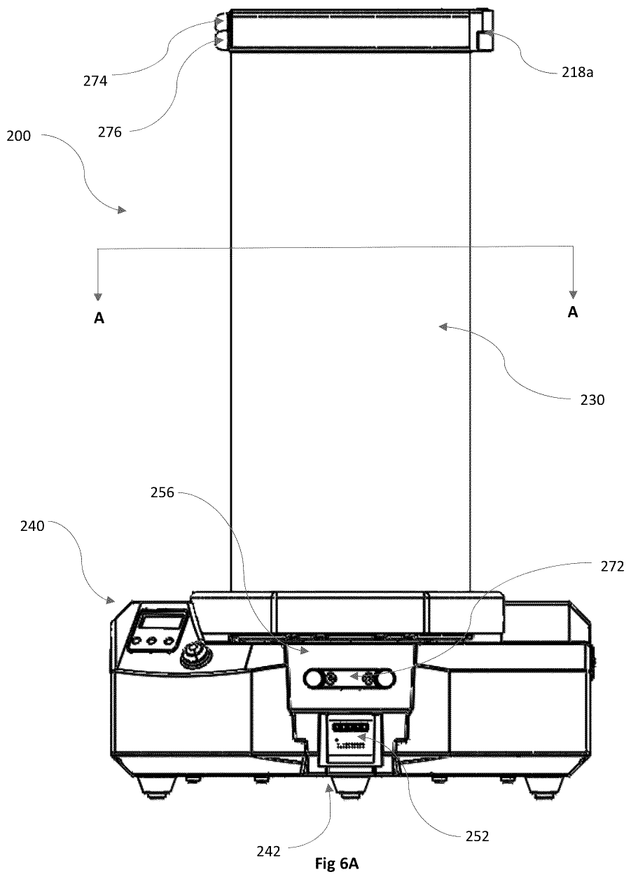

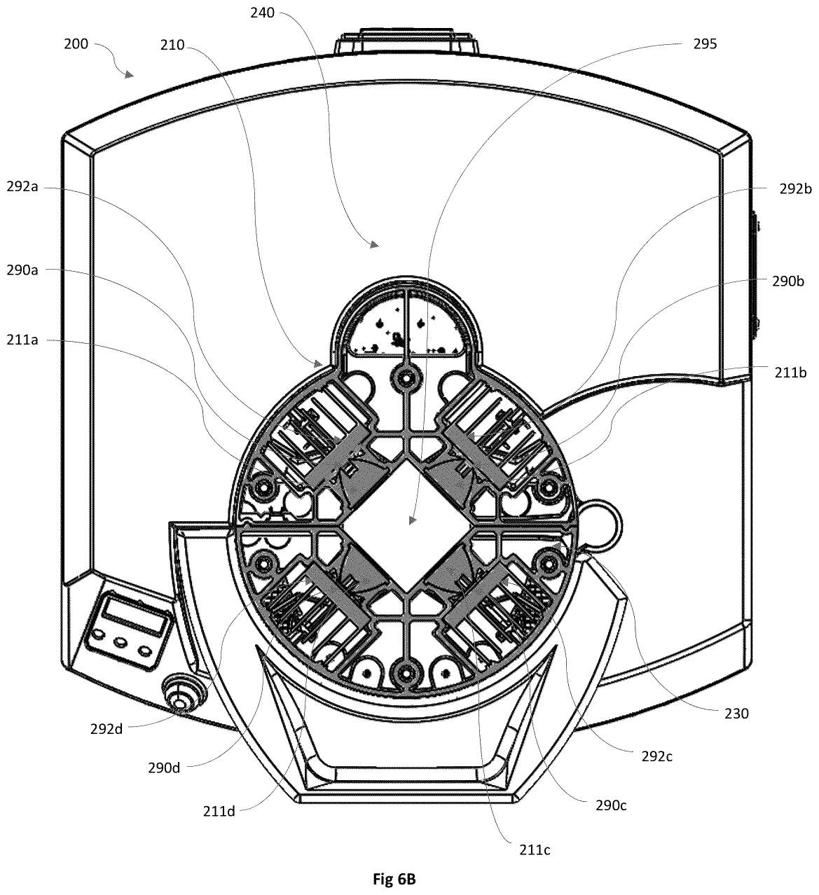

FIG. 6A illustrates a front view of the light chamber 200 in the closed position. On a back face 256 of the docking bay 242 is an electrical connector 252 and a connection mechanism 272. The electrical connector 252 can provide power and control signals to the vein carrier 300 and receive electrical signals from the vein carrier 300. The connection mechanism 272 is configured to securely hold the lower support 310 of the vein carrier 300 in the docking bay 242. The connection mechanism 272 can be a magnet, a magnetic material (e.g., a metal), or other common connection mechanisms such as but not limited to a latch, a detent, a snap-fit, and the like. The vein carrier 300 can have an opposite connection mechanism on the lower support 310 of the vein carrier 300 configured to connect to the connection mechanism 272 of the of the docking bay 242.

FIG. 6B illustrates a cross sectional view through line A-A of FIG. 6A of the light chamber 200 through the fixed light tower 220 and the moveable light tower 230. The fixed light tower 220 and the moveable light tower 230 can have one or more light source assemblies. The light source assemblies can include one or more light sources and one or more lenses. The fixed light tower 220 can have two light sources 290(a), 290(b). In other aspects, the fixed light tower 220 can have one, two, three, four, five, six, seven, eight, nine, ten, or more light sources. The moveable light tower 230 can have two light sources 290(c), 290(d). In other aspects, the moveable light tower 230 can have one, two, three, four, five, six, seven, eight, nine, ten, or more light sources. As illustrated in FIG. 6B, the lenses 211(a), 211(b), 211(c), 211(d) can have a parabolic profile. The parabolic profile of the lenses 211(a), 211(b), 211(c), 211(d) can direct light emitted in a conical pattern from the light sources 290(a), 290(b), 290(c), 290(d) in the direction of a center point 295 of the fixed light tower 220 and the moveable light tower 230 (e.g., the center of the vein carrier 300 when the vein carrier 300 is installed), thereby providing light to the entire surface area of the vein. The lenses 211(a), 211(b), 211(c), 211(d) can be made from any material configured for transmitting light including, but not limited to, polycarbonate, quartz, borosilicate glass, and the like. It will be appreciated that other configurations and numbers of lenses and light sources can be used in the tissue modification system 100.

In an aspect, the light sources 290(a), 290(b), 290(c), 290(d) can be LEDs, halogen bulbs, mercury vapor bulbs, and the like. In an aspect, the light sources 290(a), 290(b), 290(c), 290(d) can be green LEDs with a spectrum of about 495 nm to about 570 nm. In an aspect, the light sources can be green LEDs with a spectrum of about 530 nm to about 540 nm, or about 532 nm. In another aspect, the light sources 290(a), 290(b), 290(c), 290(d) can be laser diodes configured to emit light (e.g., laser beams) at a wavelength of about 800 nm to about 850 nm, about 850 nm to about 900 nm, about 900 nm to about 950 nm, about 950 nm to about 1000 nm, about 1000 nm to about 1050 nm, about 1050 nm to about 1100 nm. In another aspect, the light sources 290(a), 290(b), 290(c), 290(d) can be other types of lasers configured to emit wavelengths between ultraviolet, visible, or near infrared, or infrared light. The lasers can emit laser beams at wavelengths between about 180 nm to about 1,200 nm, including any value therebetween. It will be appreciated that other types of light sources with different spectrums can be used in the tissue modification system 100. In an aspect, the light sources 290(a), 290(b), 290(c), 290(d) can be configured such that the light sources 290(a), 290(b), 290(c), 290(d) cannot be turned on when the light chamber 200 is in the open configuration (e.g., the moveable light tower 230 is not removably coupled to the fixed light tower). The lights 290(a), 290(b), 290(c), 290(d) can be electronically wired such that the lights 290(a), 290(b), 290(c), 290(d) will not turn on when the light chamber is in the open configuration. In another aspect, the light sources 290(a), 290(b), 290(c), 290(d) can be controlled by a control system that prevents the light sources 290(a), 290(b), 290(c), 290(d) from turning on when the light chamber 200 is in the open configuration.

As illustrated in FIG. 6B, the light chamber 200 can include one or more heat sinks. In some aspects, the light sources 290(a), 290(b), 290(c), 290(d) can be backed by (e.g., coupled to) heat sinks 292(a), 292(b), 292(c), 292(d), respectively. The heat sinks 292(a), 292(b), 292(c), 292(d) can conduct heat away from the light sources 290(a), 290(b), 290(c), 290(d) to keep the light sources 290(a), 290(b), 290(c), 290(d) operating at a safe temperature. The heat sinks 292(a), 292(b), 292(c), 292(d) can be made from any high thermal conductive material such as copper, aluminum, and the like. The heat sinks 292(a), 292(b), 292(c), 292(d) can include multiple fins to create a large surface area for cooling. For example, the heat sinks 292(a), 292(b), 292(c), 292(d) can have two fins to one thousand fins, or more. The fins can have any length or geometry for dissipating heat. As discussed above, the air flow provided through the vents in the bottom bases 217, 237 and/or the top caps 212, 232 can be directed through the heat sinks 292(a), 292(b), 292(c), 292(d) to dissipate heat and allow the light sources 290(a), 290(b), 290(c), 290(d) to operate at a safe temperature. In other configurations with more or less light sources, each light source can be backed by (e.g., coupled to) a respective heat sink. It will be appreciated that any number of light sources, lenses, and heat sinks can be used. The light sources and lenses can be configured to ensure that the outer surface of a vein placed at the center point 295 in the vein carrier 300 is completely covered by the light emitted by the light sources either simultaneously or at different times. The heat sinks can be configured to maintain a safe operating temperature of the one or more light sources and the light chamber 200.

In an aspect, the one or more fans or air flow providers (e.g., pressurized air flow provider and the like) in the support base 240, the openings 250(a), 250(b), the one or more vents in the bottom bases 217, 237, the heat sinks 292(a), 292(b), 292(c), 292(d), and the one or more vents 215(a), 215(b), 215(c), 235(a), 235(b), 235(c) in the top caps 212, 232 can be referred to as a light chamber temperature control assembly. The light chamber temperature control assembly can be in fluid communication with the light chamber 200. Air flow can be provided by the fan or another air flow provider in the support base. The light chamber temperature control assembly can further include additional fans, openings, heat sinks, cents, and other components. The air flow can flow through the openings 250(a), 250(b) in the support base 240, through the one or more vents in the bottom bases 217, 237, through the heat sinks 292(a), 292(b), 292(c), 292(d), and out through the one or more vents 215(a), 215(b), 215(c), 235(a), 235(b), 235(c) in the top caps 212, 232. The light chamber temperature control assembly can cool the light sources 290(a), 290(b), 290(c), 290(d). In an aspect, the top caps 212, 232 can have a second fan configured to draw air out of the light chamber 200 through the one or more vents 215(a), 215(b), 215(c), 235(a), 235(b), 235(c). The light chamber temperature control assembly can be configured to provide air flow the entire time the light chamber 200 is in the closed configuration and/or for desired times regardless of the configuration (e.g., open or closed) of the light chamber 200.

The light chamber 200 can further include a control system such as, but not limited to, a processor, microprocessor, controller, microcontroller, CPU, and the like. The control system can control power delivery to the light sources 290(a), 290(b), 290(c), 290(d) and can be used to measure both current draw and temperature of the light sources 290(a), 290(b), 290(c), 290(d). The control system can ensure that the light sources 290(a), 290(b), 290(c), 290(d) are activated as intended by measuring the current draw and temperature. The control system can also control power delivery to the vein carrier 300 through the electrical connector 252. The control system can receive and interpret data from the vein carrier 300 to modify power delivery. The control system can also control the temperature in the light chamber 200 by controlling the fan speed or air flow rate in the light chamber temperature control assembly. The control system can also be operable to shut the light sources 290(a), 290(b), 290(c), 290(d) off when the light chamber 200 is in the open configuration (e.g., the movable light tower 230 is not coupled to the fixed light tower 220). The control system can further be configured to automate any of the movement of attachment mechanisms described herein via a robotic arm, automating the components using mechanical motors, or the like.

The control system can be in electronic communication with an internal modem. The internal modem can upload data related to the operation of system such as light parameters, temperatures, humidity, treatment parameters, durations, and other data to one or more servers. In some aspects, the one or more servers can include a cloud computing network. The one or more servers can be monitored to ensure correct and safe operation of the tissue modification system 100.

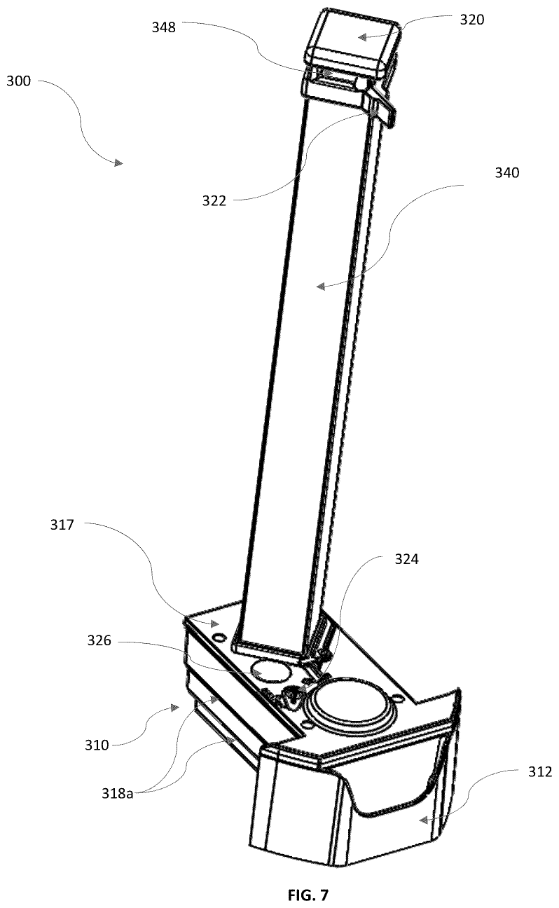

FIG. 7 illustrates the vein carrier 300. The top portion of the vein carrier 300 can be covered by a cover 320 that prevents ingress of any particles or materials from above into the inside of the vein carrier 300 (e.g., translucent holder 340). The cover 320 can be offset from the translucent holder 340 by a venting gap 348. The venting gap 348 can allow air and moisture to exit the top of the translucent holder 340. The lower support 310 of the vein carrier 300 can include a fluid port 324 on the upper surface 317. The fluid port 324 can be used to add fluid such as, but not limited to, saline, water, lactated ringers' solution, and the like. The fluid port 324 can be open to the outside at the upper surface 317. In some aspects, the fluid port 324 can include a cover that is removed from the fluid port 324 to add fluid and then placed back over the fluid port 324 to ensure the fluid stays inside the lower support 310. The cover of the fluid port 324 can prevent exterior particles or materials from entering the fluid port. In another aspect, the fluid port 324 cover can be a filter operable to receive fluids but prevent the ingress of exterior particles and materials. The upper surface 317 of the lower support 310 can also include a gauge window 326. The gauge window 326 can be used to visualize the amount of fluid that has been added to the lower support. In another aspect, the fluid port can include a volume sensor and/or flow sensor in communication with the control system and the indicator screen 244, such that the indicator screen can display an amount of fluid within the lower support.

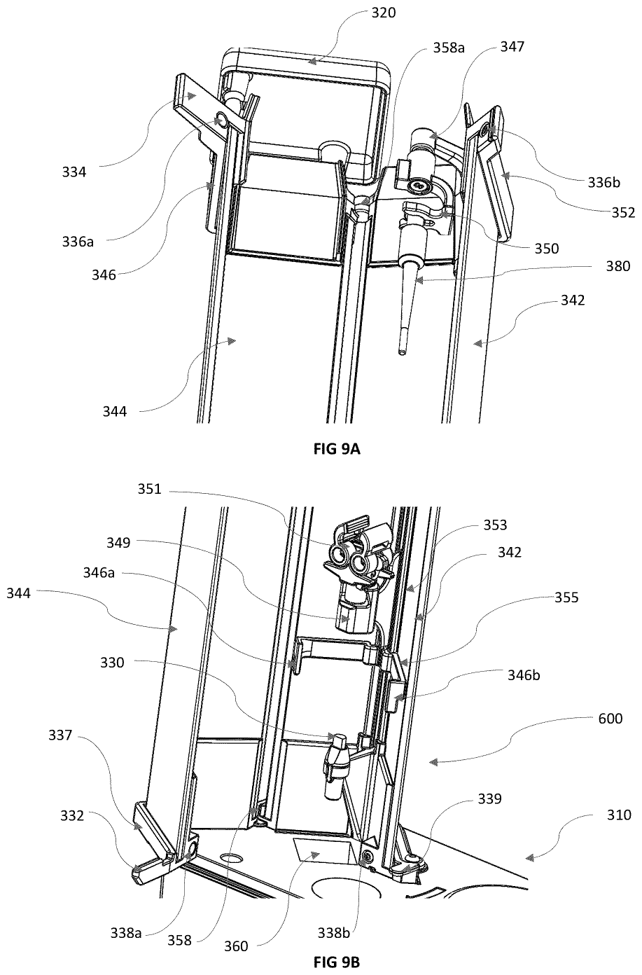

FIGS. 8, 9A, and 9B illustrate the vein carrier in an open configuration. The open configuration of the vein carrier can allow a vein to be inserted and/or removed. The upper translucent holder 340 can include a fixed translucent section 342 and a moveable translucent section 344. The fixed translucent section 342 can be attached to the lower support 310 with a lower bracket 339, as illustrated in FIG. 9B. The lower bracket 339 can include portion of a lower connecting hinge 358(b). The other portion of the lower connecting hinge 358(b) can be connected to a lower moveable bracket 337 on the moveable translucent section 344. The lower moveable bracket 337 can support the moveable translucent section 344. The top of the fixed translucent section 342 can include an upper bracket 352 configured to cap the top of the fixed translucent section 342. The upper bracket 352 can include a portion of an upper connecting hinge 358(a). The other portion of the upper connecting hinge 358(a) can be connected to an upper moveable bracket 346 which caps the top of the moveable translucent section 344. Using the lower connecting hinge 358(b) and the upper connecting hinge 358(a), the moveable translucent section 344 can be pivoted away from the fixed translucent section 342 in order to access the interior of the upper translucent holder 340 (e.g., to attach a vein or biological component to the upper translucent holder 340).

As illustrated in FIG. 9A, the upper moveable bracket 346 can include an upper extension tab 334 and the lower moveable bracket 337 can include a lower extension tab 332. The upper extension tab 334 and the lower extension tab 332 can be configured to enable the user to move the movable translucent section 344 relative to the fixed translucent section 342 to open and close the translucent holder 340. In some aspects, the fixed translucent section 342 can be removably coupled to the moveable translucent section 344 via one or more attachment mechanisms. In some aspects, the one or more attachment mechanisms can include one or more magnetic materials, one or more snap-fit connectors, one or more laches, and/or any other suitable attachment mechanism.

As illustrated in FIG. 9A, the upper moveable bracket 346 can include a magnet magnetic material 336(a) and the upper bracket 352 includes a magnetic material 336(b). In an aspect, one of the magnetic materials 336(a), 336(b) can be a magnet while the other magnetic material can be a metal (e.g., iron, steel, stainless steel, and the like). In another aspect, both of the magnetic materials 336(a), 336(b) can be magnets having opposite polarities. The magnetic materials 336(a), 336(b) can be configured to removably couple to one another. In yet another aspect, the magnetic materials 336(a), 336(b) can be replaced with other connection mechanisms such as snap-fits, latches, screw and hole configurations, and the like.

As illustrated in FIG. 9B, the lower moveable bracket 337 can include a magnetic material 338(a) and the lower bracket 339 can include a magnetic material 338(b). In an aspect, one of the magnetic materials 338(a), 338(b) can be a magnet while the other magnetic material can be a metal (e.g., iron, steel, stainless steel, and the like). In another aspect, both of the magnetic materials 338(a), 338(b) can be magnets having opposite polarities. The magnetic materials 338(a), 338(b) can be configured to removably couple to one another. The magnetic material magnet 336(a) and the magnetic material 338(a) of the fixed translucent section 342 can be configured to couple to the magnetic material 336(b) and the magnetic material 338(b) of the moveable translucent section 344 to hold the moveable translucent section 344 against the fixed translucent section 342 when the vein carrier 300 is in a closed configuration. In yet another aspect, the magnetic materials 338(a), 338(b) can be replaced with other connection mechanisms such as snap-fits, latches, screw and hole configurations, and the like.

In another aspect, the moveable translucent section 344 and the fixed translucent section 342 can have opposing locking mechanisms that lock when the translucent holder 340 is in the closed configuration (e.g., the moveable translucent section 344 and the fixed translucent section 342 are removably coupled). The locking mechanisms can be any of the connection mechanism described herein. The locking mechanisms can be any type of lock operable to be actuated by the control system when treatment (e.g., light) is being delivered to the vein in the vein carrier 300.

In an aspect, the magnetic materials 336(a), 336(b), 338(a), 338(b) can have sensors configured to provide information to the control system to ensure that the fixed translucent section 342 is properly coupled to the moveable translucent section 344. Similarly, any other connection mechanisms described herein can have sensors in communication with the control system to ensure proper connections. In some aspects, the sensors can include one or more location sensors and/or one or more contact sensors.

The upper moveable bracket 346 and the lower moveable bracket 337 can be connected to the upper bracket 352 and the lower bracket 339 by means other than the upper hinge 358(a) and the lower hinge 358(b). For example, the upper moveable bracket 346 and the lower moveable bracket 337 can be connected to the upper bracket 352 and the lower bracket 339 with screws, latches, magnets or other mechanical fasteners.

As illustrated in FIG. 8, the upper surface 317 of the lower support 310 can include a cooling vent 360 that connects the inside of the upper translucent holder 340 to the inside of the lower support 310 (e.g., fluid communication). In another aspect, the upper surface of the lower support 310 can include more than one vent. The cooling vent 360 can be an unobstructed opening (e.g., window), as illustrated in FIGS. 8 and 9B. The cooling vent 360 can be covered by a grate, screen, or filter to prevent any undesired material or particles from entering the inside of the lower support 310.

The translucent holder 340 can also include one or more sensors. In some aspects, the one or more sensors can be disposed within the translucent holder 340. As illustrated in FIG. 8, the one or more sensors 330 can be positioned on the lower bracket 339 of the fixed translucent section 342. The one or more sensors 330 can be positioned at the center point 295 of light chamber 200 when the vein carrier 300 is docked with the light chamber 200 in the closed configuration. The one or more sensors 330 can include any type of sensor including, but not limited to, one or more temperature sensors (e.g., thermocouple or thermistor), one or more light sensors (e.g., photodetectors), one or more humidity sensors (e.g., hygrometer), and/or other sensors for measuring and recording information within the translucent holder 340 of the vein carrier 300.

In an aspect, the translucent holder can include one or more location sensors and/or contact sensors to determine that the fixed translucent section 342 is properly coupled to the moveable translucent section 344 before treatment begins. In an aspect, the lower bracket 339 can include a temperature sensor, a light sensor, and a humidity sensor. In another aspect, the one or more sensors 330 can be positioned on the upper bracket 352 or a floating bracket 355. In other aspects, the one or more sensors 330 can be attached to each of the lower bracket 339, the upper bracket 352, and the floating bracket 355. It will be appreciated that the one or more sensors 330 can also be positioned on the upper moveable bracket 346 and the lower moveable bracket 337 and/or other positions within the translucent holder 340. The one or more sensors 330 can be used to monitor the environment in the translucent holder 340 and provide data to the light chamber 200, via the control system, such that the data is recorded for analysis. In some aspects, the data (e.g., measurements) from the one or more sensors 330 can be displayed on the indicator screen 244 or transmitted to another display screen. The data from the one or more sensors 330 can be used to adjust the treatment parameters of the tissue modification system 100 to optimize and/or improve the treatment. In some aspects, the control system can automatically adjust the parameters (e.g., fan speed, humidifier operation, and/light parameters) to provide the desired treatment.

In some aspects, the indicator screen 244 can display warnings, alerts, and/or errors related to the one or more sensors 330. The indicator screen 244 can display visual and/or audible warnings if temperature exceeds or falls below a threshold temperature range, if humidity exceeds or falls below a threshold humidity range, if light delivery is not sufficient or exceeds a threshold, if components are not properly connected (e.g., coupled) or other warnings.

In some aspects, the vein carrier 300 can include an adjustable length vein attachment mechanism for mounting a vein in the vein carrier 300. The adjustable length vein attachment mechanism can include a vessel cannula 380, an upper attachment portion operable to secure an end of the vein connected to the vessel cannula 380, and a lower adjustable length attachment portion operable to secure an opposite end of the vein (e.g., lower end).

As illustrated in FIGS. 8 and 9A, the upper bracket 352 of the fixed translucent section 342 can include an upper magnetic holder 347 configured to secure a vessel cannula clip 350. The vessel cannula clip 350 can be configured to hold a vessel cannula 380. The vessel cannula 380 can be used in by-pass surgeries to cannulate the veins that are removed from the patient. When veins are removed from the patient, a surgical team can insert the vessel cannula 380 in a proximal end of the vein and secure the vessel cannula 380 with sutures to the vein. The vessel cannula 380 can be used to flush the inside of the vein to ensure there is no blood or coagulated blood clogging the vein. The vessel cannula 380 can also be used to pressurize the vein to ensure there are no fluid leaks or weak sections of the vein wall both of which are undesirable for a vein being used in by-pass procedures. As the vein is already attached to a vessel cannula 380, the vessel cannula clip 350 can be used to secure the vein to the inside of the translucent holder 340. The upper magnetic holder 347 can include a magnet or a magnetic material. The vessel cannula clip 350 can include a magnet and/or magnetic material such that the vessel cannula clip 350 can be quickly and easily attached and detached from the upper magnetic holder 347. In other aspects, other attachment mechanisms can be used to attach the vessel cannula clip 350 to an upper holder. For example, a latch, a detent, a snap-fit, and the like can be used to attach the cannula clip 350 to the upper holder.

As illustrated in FIG. 9B, the fixed translucent section 342 can include a guide rail 353. The guide rail can span from the bottom of the fixed translucent section 342 to the top of the fixed translucent section 342. A floating bracket 355 can be attached (e.g., moveably coupled) to the guide rail 353 such that the floating bracket 355 can translate vertically (e.g., up and down) within the translucent holder 340 along the guide rail 353. The floating bracket 355 can include a lower magnetic holder 349 that is configured to secure a vein clamp 351. The lower magnetic holder 349 can be a magnet or magnetic material. The vein clamp 351 can include a magnet and/or magnetic material such that the vein clamp 351 can be quickly and easily attached and detached from the lower magnetic holder 349. In other aspects, other attachment mechanisms such as, but not limited to, a latch, a detent, a snap-fit, and the like can be used to secure the vein clamp 351 to the floating bracket. The vein clamp 351 can be attached to the vein at an opposite end from the vessel cannula 380 (e.g., distal end of the vein). The vein clamp 351 can be configured in any type of clamping configuration including but not limited to angularly pivoting jaw clamps, linear translating jaw clamps, annular constricting clamps (Touhy-Borst), and the like.

The floating bracket 355 can translate along the guide rail 353, thereby providing an adjustable length depending on the length of the vein. In some aspects, the vein can have a first end (proximal end) and a second end (distal end) defining a length. The first end can be secured to the vessel cannula 380 and the second end can be secured to the vein clamp 351. The first end and the second end of the vein can define a vein length. The vein length can be about 5 cm to about 10 cm, about 10 cm to about 20 cm, about 20 cm to about 30 cm, about 30 cm to about 40 cm, about 40 cm to about 50 cm, or more can be secured in the translucent holder 340. In another aspect, the vein length can be about 5 cm to about 40 cm. The vein can be held in a straight (e.g., vertical) configuration.

As illustrated in FIG. 9B, the floating bracket 355 can include safety tabs 346(a), 346(b). The safety tabs 346(a), 346(b) can extend to the outer edges of the fixed translucent section 342. When the floating bracket 355 is not attached to a vein for treatment, the floating bracket 355 can rest at the bottom of the guide rail 353 against the upper surface 317 of the lower support 310. When the floating bracket 355 is resting at the bottom of the guide rail 353, the safety tabs 346(a), 346(b) can prevent the moveable translucent section 344 from contacting the fixed translucent section 342 to close the upper translucent holder 340 due to interference with the inside of the lower moveable bracket 337. To place the moveable translucent section 344 against the fixed translucent section 342 and close the upper translucent holder 340, the floating bracket 355 can be lifted up such that the safety tabs 346(a), 346(b) are above the inside of the lower moveable bracket 337. By attaching a vein to the translucent holder 340 with the vessel cannula clip 350 secured to the upper magnetic holder 347 and the vein clamp 351 secured to the lower magnetic holder 349, the floating bracket 355 and therefore the safety tabs 346(a), 346(b) are lifted above the inside of the lower moveable bracket 337. In this manner, the safety tabs 346(a), 346(b) ensure that the translucent holder 340 cannot be closed and a treatment cannot be performed unless there is a vein held in the upper translucent holder 340 and the length of the vein being treated is such that the entirety of the vein is within the translucent section which receives light. Therefore, the length of vein being held in the vein carrier 300 does not extend below the top of the inside of the lower moveable bracket 337, which would prevent the light from reaching that portion of the vein.

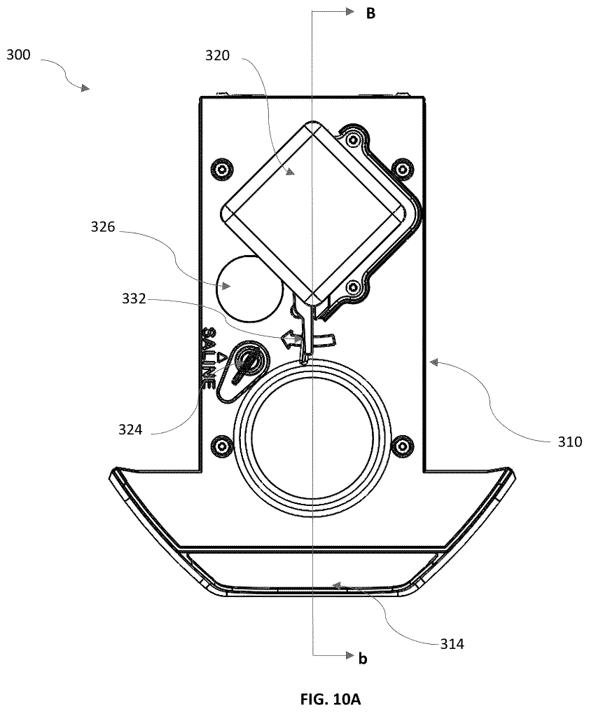

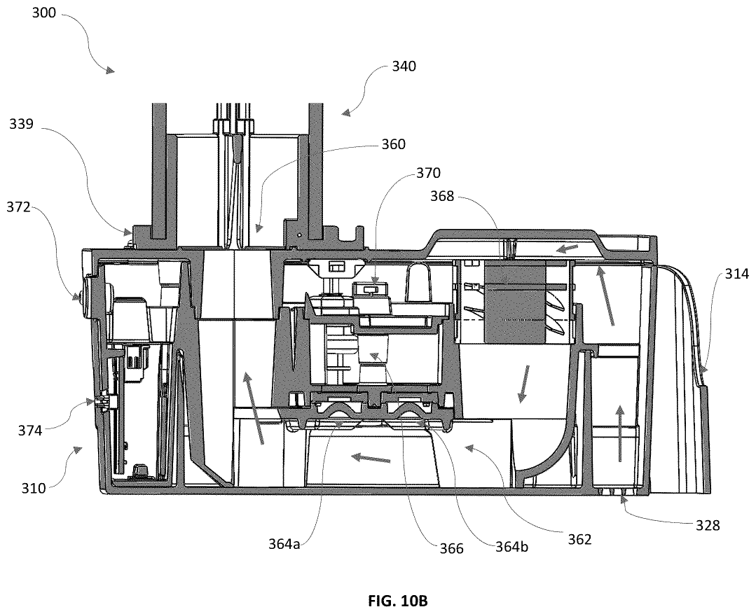

FIG. 10B illustrates a cross section through line B-B of FIG. 10A of the lower support 310 of the vein carrier 300. The lower support 310 (e.g., rear portion that connects to the docking bay 242) can include an electrical carrier connector 374 configured to connect to the electrical connector 252 of the light chamber 200. The electrical carrier connector 374 can be configured to derive power and control signals form the light chamber 200 to operate the vein carrier 300. The electrical carrier connector 374 can also be configured to provide data (e.g., measurements from one or more sensors 330 and/or other sensors) from the vein carrier 300 to the light chamber 200. For example, when the electrical carrier connector 374 is connected to the electrical connector 252 of the light chamber 200, the indicator screen 244 can display a proper connection (e.g., visual indication) between the vein carrier 300 and the light chamber 200. The data from the one or more sensors 330 can also be provided to the light chamber 200, and thereby the control system and the indicator screen 244, via the electrical carrier connector 374 and the electrical connector 252. Further, data related to the operation of a carrier fan 368 can be provided to the light chamber 200, and thereby the control system and the indicator screen 244, via the electrical carrier connector 374 and the electrical connector 252. The carrier fan 368 can also be controlled by the control system.

As illustrated in FIG. 10B, the rear end of the lower support 310 can also include a carrier connector 372 configured to engage with the connection mechanism 272 on the light chamber 200 to secure the vein carrier to the light chamber 200 and ensure the electrical carrier connector 374 is engaged with the electrical connector 252. The lower support 310 can also include a vein carrier temperature control assembly. The vein carrier temperature control assembly can include a carrier fan 368, an air intake 328, a cooling vent 360, an air channel 362, one or more humidifiers 364(a), 364(b), and a fluid chamber 366. The vein carrier temperature control assembly can be in fluid communication with the translucent holder 340. The lower support 310 can include a carrier fan 368 configured to draw air into the lower support 310 through the air intake 328 and push air through the air channel 362 and out the cooling vent 360 on a path shown by the arrows of FIG. 10B. The lower support 310 can also include a fluid chamber 366 which can be filled by the user adding fluid though the fluid port 324. The fluid chamber 366 can include one or more fluid sensors 370 that can detect the overall fluid level and determine whether a sufficient amount of fluid is available to treat a vein. The one or more fluid sensors can provide information to the control system and the indicator screen 244 can display information related to the fluid level. For example, the indicator screen 244 can display that sufficient fluid is available or additional fluid needs to be added to the fluid chamber 366. The vein carrier temperature control assembly can also include one or more additional fans, vents, fluid chambers, humidifiers, air intakes, air channels, and other components.

As illustrated in FIG. 10B, the lower support 310 can contain one or more humidifiers 364(a), 364(b) that can be a part of the temperature control system. In an aspect, the humidifiers 364(a), 364(b) can be ultrasonic humidifiers. The humidifiers 364(a), 364(b) can be configured to aerosolize the fluid in the fluid chamber 366 and add the water vapor to the air passing through the air channel 362. The humidifiers 364(a), 364(b) can include piezoelectric transducers to create a high frequency mechanical oscillation in the fluid. The carrier fan 368 and the humidifiers 364(a), 364(b) can provide humidified air flowing through the translucent holder 340 to keep the vein cool and moist during treatment. In other aspects, more than one fan and more than two humidifiers can be used. In an aspect, the translucent holder 340 can have one or more vents (e.g., venting gap 348) near or on the cover 320 configured to allow air to flow out of the translucent holder 340. In another aspect, the cover 320 can include a second fan configured to draw air out through the one or more vents (e.g., air outtake vents) on the cover 320. It will be appreciated that any number or configuration of fans, humidifiers, fluid chambers, air intakes, and vents can be used to keep the vein cool and moist during treatment. The control system can control the operation of the vein carrier temperature control system. In an aspect, the control system can maintain the humidity level and temperature of the translucent holder 340 to a desired temperature and humidity. In an aspect, the temperature control system can operate the entire time the vein carrier 300 docked to the light chamber 200. In another aspect, the fan 368 and/or humidifiers 346(a), 346(b) can be actuated based on the temperature and/or humidity in the translucent holder 340. For example, the fan 368 and/or humidifiers 346(a), 346(b) can be turned on when the temperature and/or humidity is outside of a desired range. When the temperature and/or humidity is within a desired range, the fan 368 and/or humidifiers can be turned off.

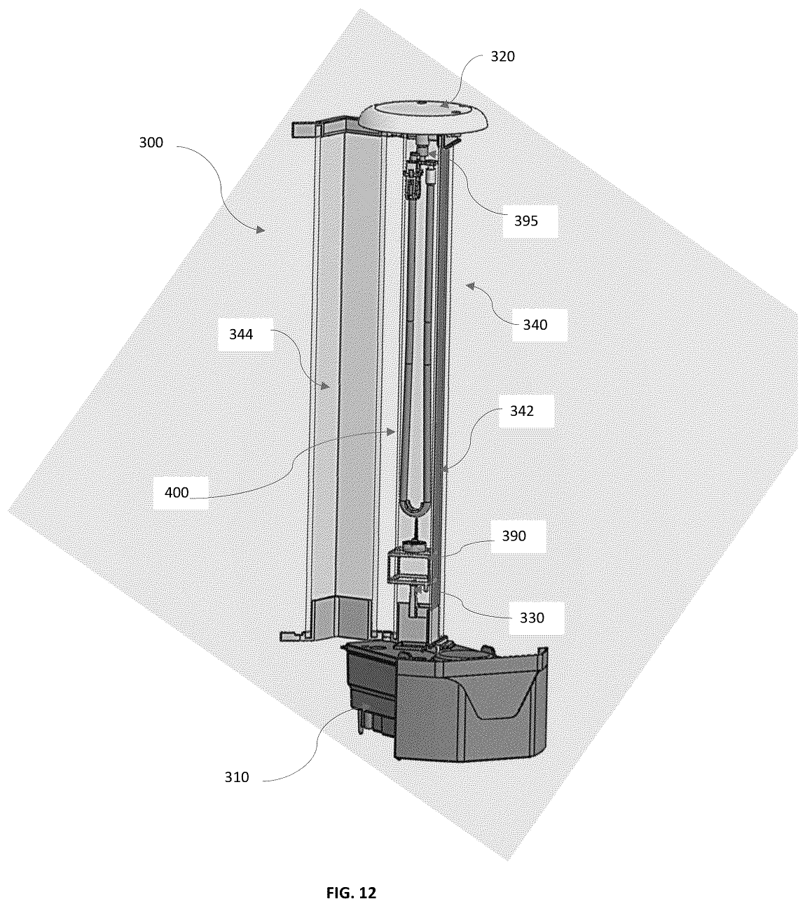

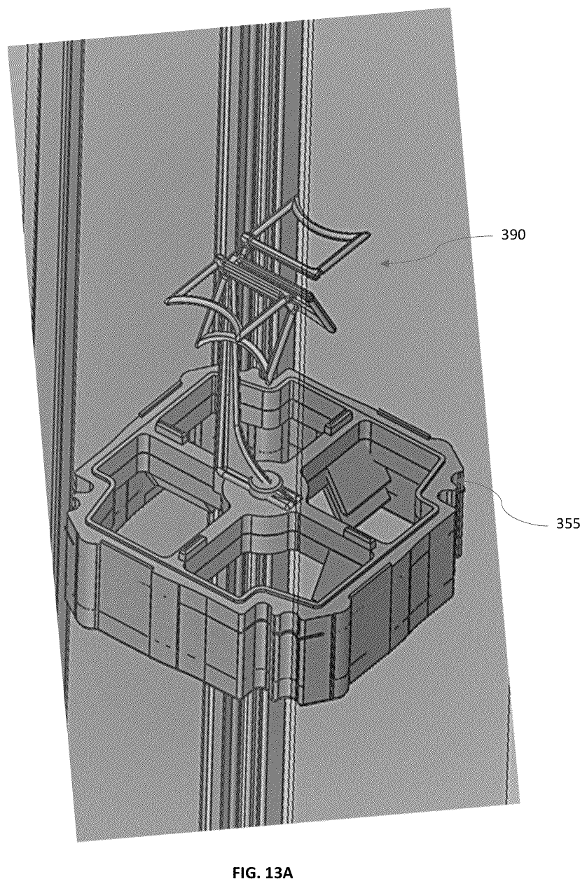

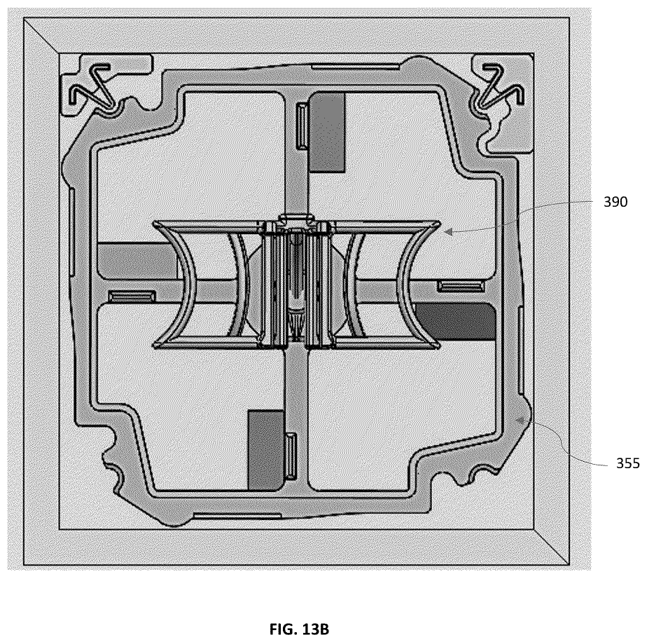

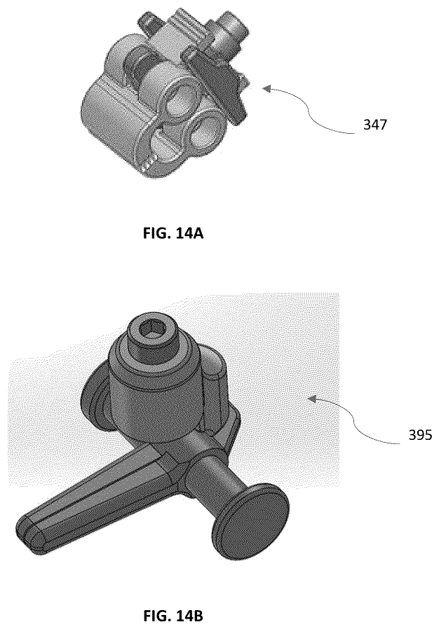

As illustrated in FIG. 12, the tissue modification system 100 can be used to treat tissue samples that are longer than the height of the translucent holder 340 by looping the tissue sample (e.g., vein) within the translucent holder 340. The vein carrier 300 can include a semi-circular holder 390 secured to the floating bracket 355. A vessel cannula can be used to hold one end of the vein, as described above, and a vein clamp can secure the other end of the vein, as described above. The vessel cannula and vein clamp holder 395 can be secured to the upper magnetic holder 347. FIG. 14A illustrates the magnetic holder 347 for the vessel cannula and the vein clamp holder 395. FIG. 14B illustrates the vein clamp holder 395. In another aspect, two vessel cannulas can be connected to the vein, one on each end. The vein can hang in a “U” shape inside the translucent holder 340. The bottom of the “U” of the vein (e.g., the intermediate portions of the vein between the two ends of the vein) is contained by the semi-circular holder 390 that separates the two sides of the “U” and holds the inner curve of the bottom of the “U” open, as illustrated in FIGS. 13A-13B. The semi-circular holder 390 can have multiple arms to curve the vein into the “U” shape. As illustrated in FIGS. 13A-13B, the semi-circular holder 390 can have four arms each having an opening configured to receive a portion of the vein and contain the vein. By separating the two sides of the “U” and holding the inner curve of the bottom of the “U” open, light from the light sources 290(a), 290(b), 290(c), 290(d) can reach all of the outer surfaces of the vein to modify the tissue. Using the semi-circular holder 390 allows the tissue modification system to treat vein samples that are approximately twice the length of the height of the translucent holder 340 with the veins in a “U” configuration 400.

The tissue modification system 100 is operable to modify a photo-initiator coated tissue sample secured in the vein carrier 300 docked in the light chamber 200. Modification is conducted by activating the light sources 290(a), 290(b), 290(c), 290(d) of the light chamber 200. The light from the light sources 290(a), 290(b), 290(c), 290(d) is directed by the lenses 211(a), 211(b), 211(c), 211(d) though the translucent holder 340 to the tissue sample (e.g., vein) that is contained inside the translucent holder 340. The light can excite the photo-initiator and creates singlet and triplet oxygen states. These oxygen states can crosslink with the collagen in the tissue sample to create stable covalent protein photo crosslinking in the collagen matrix. Covalent protein photo crosslinking can stiffen the tissue and increase tissue strength, thereby modifying the tissue sample.

In some aspects, the photo-initiator can be applied to the vein before the vein is secured in the vein carrier 300. In some aspects, the vein carrier 300 can include one or more spraying manifolds in communication with a pressure source and a photo-initiator source. The vein can be secured within the vein carrier 300 and the spraying manifolds can spray the vein with the photo-initiator. In some examples, the pressure source moves the photo-initiator from the photo-initiator source and out through the one or more spraying manifolds.

When the light sources 290(a), 290(b), 290(c), 290(d) are activated excess heat can be generated. When the light from the light sources 290(a), 290(b), 290(c), 290(d) is absorbed by the photo-initiator and the tissue sample excess heat can be generated. The tissue sample, having been recently removed from the patient's body, is viable tissue and needs to remain viable tissue if it is to be anastomosed and re-implanted back into the patient's body after treatment by the tissue modification system 100. Viable tissue can only withstand temperatures above body temperature for a limited amount of time. The higher the temperature the shorter the time period the tissue can withstand and still remain viable. A safe elevated temperature for the tissue to undergo during treatment by the tissue modification system 100 is 40 degrees Celsius (or less). In order to ensure that the tissue sample is not subjected to temperatures above 40 degrees Celsius during treatment, the light chamber 200 includes a fan and heatsinks 292(a), 292(b), 292(c), 292(d) to keep the light sources 290(a), 290(b), 290(c), 290(d) as cool as possible. Keeping the light sources 290(a), 290(b), 290(c), 290(d) cool helps to keep the overall tissue modification system 100 cool and thereby the tissue sample at 40 degrees Celsius or less. In addition, to ensure the tissue sample is not subjected to temperatures above 40 degrees Celsius during treatment, the vein carrier 300 includes the carrier fan 368 to provide air flow through the translucent holder 340 past the tissue sample. The vein carrier 300 also includes one or more sensors 330 that can be used to measure the temperature in the translucent holder 340 to ensure the temperature does not rise above 40 degrees Celsius.

The tissue modification system 100 can alter treatment parameters to ensure the temperature does not rise above 40 degrees Celsius. For example, the light chamber 200 can reduce the current driving the light sources 290(a), 290(b), 290(c), 290(d) to reduce the heat produced by the light sources 290(a), 290(b), 290(c), 290(d) and/or to reduce the light output that can generate heat when absorbed by the sample tissue. Additionally, the tissue modification system 100 can increase the fan speed of the fan in the support base 240 of the light chamber 200 and/or the speed of the carrier fan 368 to reduce the temperature. The tissue modification system 100 can control how long the light sources 290(a), 290(b), 290(c), 290(d) are on and/or when they are on to control the temperature. The tissue modification system 100 can provide light from the light sources 290(a), 290(b), 290(c), 290(d) in a sequential or an overlapping sequential order. The light chamber 200 can turn light source 290(a) on for ten seconds at the start of light treatment then after 3⅓ seconds the light chamber 200 can turn on light source 290(b) for ten seconds, then after another 3⅓ seconds or 6⅔ seconds the light chamber 200 can turn on light source 290(c) for ten seconds, finally after ten seconds from the start (e.g., when light source 290(a) is turned off), the light chamber 200 can turn on light source 290(d) for ten seconds. After 13⅓ seconds the light chamber 200 can turn light source 290(a) back on for ten seconds, and so on. In this manner, the light chamber 200 can cycle each light source 290(a), 290(b), 290(c), 290(d) on for ten seconds and off for 3⅓ second to deliver light from only three light sources 290(a), 290(b), 290(c), 290(d) at any one time (with the fourth light source being off). In another aspect, the light sources 290(a), 290(b), 290(c), 290(d) can each be on for about 5 seconds to about 15 seconds, about 15 seconds to about 25 seconds, about 25 seconds to about 35 seconds, about 35 seconds to about 45 seconds, or more. In some aspects, the light sources 290(a), 290(b), 290(c), 290(d) can be turned on sequentially with a delay between each light source being turned on of about 1 second to about 5 seconds, thereby providing overlapping light from multiple light sources simultaneously. Controlling the on-off cycles of the light sources 290(a), 290(b), 290(c), 290(d) can also be used to control temperatures. It will be appreciated that other light source delivery intervals can be used, for example, two light sources can be on while two or off, one light source can be on while three are off, or all the light sources can be on at the same time for a short duration. The light source delivery cycle can be controlled by the control system depending on the data (e.g., temperature, humidity, etc.) from the one or more sensors 330.

During treatment the tissue modification system 100 can ensure that the sample tissue remains viable by preventing the tissue from dehydrating. The tissue modification system 100 can control power to the one or more humidifiers 364(a), 364(b) to ensure the humidity level in the translucent holder 340 is sufficient to maintain tissue moisture. The one or more sensors 330 can measure humidity levels in the translucent holder 340 and provide the data (e.g., measurements) to the tissue modification system 100 such that the tissue modification system 100 can control the humidifiers 364(a), 364(b) to ensure the sufficient humidity levels are maintained. The control system can be used to control the humidity in the translucent holder 340. The control system can increase or decrease the rate of humidity provided by the humidifiers 364(a), 364(b) to ensure that a desired humidity level is maintained in the translucent holder 340. Further, the humidity level can be displayed on the indicator screen 244. In some aspects, a humidity level of about 70% to about 75%, about 75% to about 80%, about 80% to about 85%, about 85% to about 90%, about 90% to about 95%, about 95% to about 99%, or more can be maintained in the translucent holder 340.

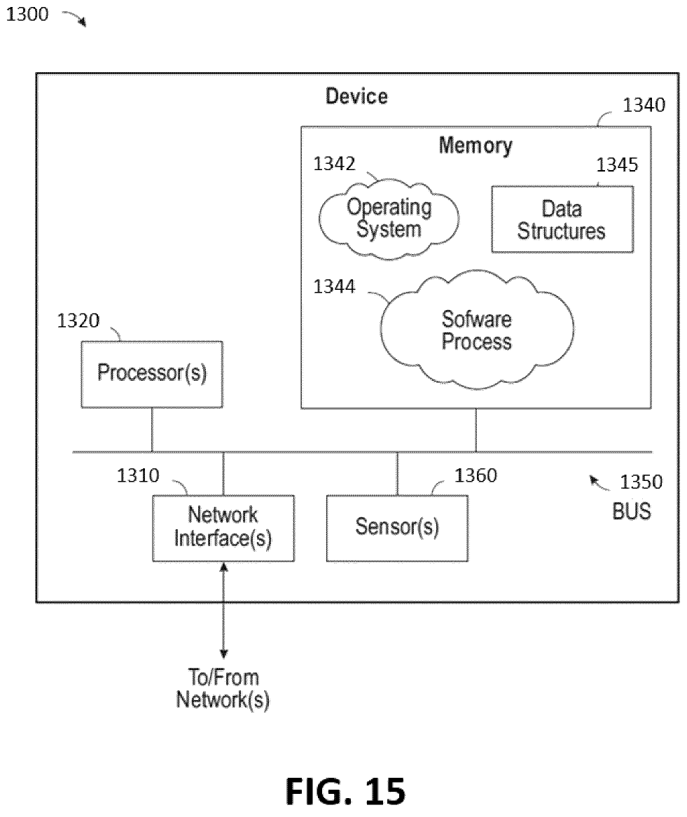

FIG. 15 is a block diagram of an exemplary controller 1300 (e.g., control system described herein). Controller 1300 is configured to perform processing of data and communicate with the various sensors, light sources, and other components described herein. In operation, controller 1300 communicates with one or more of the above-discussed components and may also be configured to communication with remote devices/systems.

As shown, controller 1300 includes hardware and software components such as network interfaces 1310, at least one processor 1320, sensors 330 (e.g., sensors for determining position of components, temperature, humidity, flow rate, volume, etc.) and a memory 1340 interconnected by a system bus 1350. Network interface(s) 1310 can include mechanical, electrical, and signaling circuitry for communicating data over communication links, which may include wired or wireless communication links. Network interfaces 1310 are configured to transmit and/or receive data using a variety of different communication protocols.

Processor 1320 represents a digital signal processor (e.g., a microprocessor, a microcontroller, or a fixed-logic processor, etc.) configured to execute instructions or logic to perform tasks for operation of the systems 100. Processor 1320 may include a general purpose processor, special-purpose processor (where software instructions are incorporated into the processor), a state machine, application specific integrated circuit (ASIC), a programmable gate array (PGA), an individual component, a distributed group of processors, and the like. Processor 1320 typically operates in conjunction with shared or dedicated hardware, including but not limited to, hardware capable of executing software and hardware. For example, processor 1320 may include elements or logic adapted to execute software programs and manipulate data structures 1345, which may reside in memory 1340.

Sensors 330, which may include sensors for temperature, humidity, contact, location, volume, flow rate, and other measurements disclosed herein, typically operate in conjunction with processor 1320 to perform measurements, and can include special-purpose processors, detectors, transmitters, receivers, and the like. In this fashion, sensors 330 may include hardware/software for generating, transmitting, receiving, detection, logging, and/or sampling various parameters of the systems 100.

Memory 1340 comprises a plurality of storage locations that are addressable by processor 1320 for storing software programs and data structures 1345 associated with the embodiments described herein. An operating system 1342, portions of which may be typically resident in memory 1340 and executed by processor 1320, functionally organizes the device by, inter alia, invoking operations in support of software processes and/or services 1344 executing on controller 1300. These software processes and/or services 1344 may perform processing of data and communication with controller 1300, as described herein. Note that while process/service 1344 is shown in centralized memory 1340, some examples provide for these processes/services to be operated in a distributed computing network.

It will be apparent to those skilled in the art that other processor and memory types, including various computer-readable media, may be used to store and execute program instructions pertaining to functions of the systems 100 described herein. Also, while the description illustrates various processes, it is expressly contemplated that various processes may be embodied as modules having portions of the process/service 1344 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 1320 or computer readable medium encoded with instructions for execution by processor 1320 that, when executed by the processor 1320, are operable to cause the processor 1320 to perform the functions described herein.

In some aspects, the systems described herein can be provided in a kit. The kit can include any one of the tissue modification systems 100 described herein and a photo-initiator. The photo-initiator can be selected from a group consisting of xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, and anthroquinone dyes. The photo-initiator used can also be selected from the group consisting of Rose Bengal (“RB”), erythrosine, riboflavin, methylene blue (MB), Toluidine Blue, Methyl Red, Janus Green B, Rhodamine B base, Nile Blue A, Nile Red, Celestine Blue, Remazol Brilliant Blue R, riboflavin-5-phosphate (R-5-P), N-hydroxypyri-dine-2-(I H)-thione (N-HTP) and photoactive derivatives thereof.

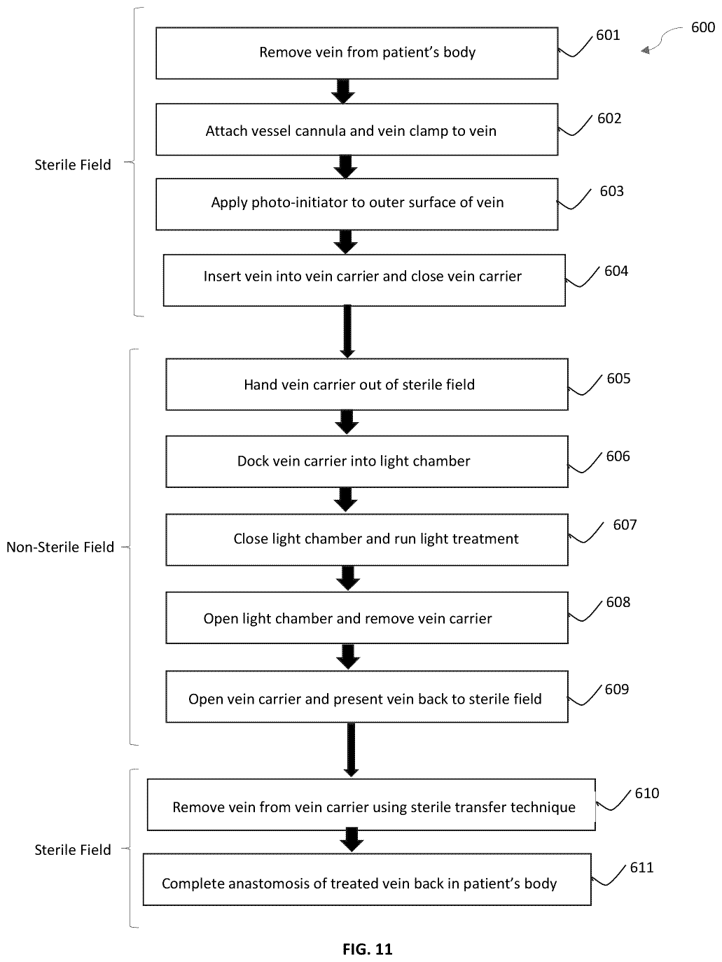

Turning now to FIG. 11, a method 600 for modification of vein tissue using a photo-initiator and light while maintaining sterility of the tissue is described. The method 600 can be conducted utilizing the systems described herein. At block 601, the method 600 can include surgically removing the vein from the patient's body. The surgical removal of the vein from the patient's body can be conducted using methods known in the art.

At block 602, the method 600 can include attaching a vessel cannula to one end of the vein and attaching a vein clamp to the other end of the vein. At block 603, the method 600 can include applying the photo-initiator to the outer surface of the vein. Applying the photo-initiator to the outer surface of the vein can be accomplished by any method used to apply a fluid substance to a surface including, but not limited to, spraying the photo-initiator on, painting it on, dipping the vein into a bath of the photo-initiator, rolling the vein over a surface coated with the photo-initiator, and the like.

The photo-initiator can be selected from a group consisting of xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, and anthroquinone dyes. The photo-initiator used can also be selected from the group consisting of Rose Bengal (“RB”), erythrosine, riboflavin, methylene blue (MB), Toluidine Blue, Methyl Red, Janus Green B, Rhodamine B base, Nile Blue A, Nile Red, Celestine Blue, Remazol Brilliant Blue R, riboflavin-5-phosphate (R-5-P), N-hydroxypyri-dine-2-(I H)-thione (N-HTP) and photoactive derivatives thereof.

At block 604, the method 600 can include inserting the coated vein into a vein carrier. In some aspects, the coated vein can be secured within the vein carrier. In some aspects, the vein carrier can include a translucent material configured to allow light to pass through the vein carrier to the coated vein. Inserting the coated vein into the vein carrier can include securing the vessel cannula holding a first end of the vein to a vessel cannula clip, securing the vessel cannula clip to an upper holder (e.g., upper magnetic holder attached to the top of a translucent holder of the vein carrier), and securing the vein clamp holding a second end of the vein to the lower holder (e.g., lower magnetic holder attached to the bottom of the translucent holder of the vein carrier). After the vein is secured in the vein carrier, a movable translucent section of the translucent holder can be placed into contact with a fixed translucent section of the translucent holder, thereby closing vein carrier with the vein secured therein. By using a sterile photo-initiator, a sterile vein carrier, a sterile fluid in the vein carrier, and by performing all of method 600 blocks 601-604 as described herein in a sterile field by an operator that is scrubbed in, the coated vein from the patient's body is now contained in the sterile vein carrier. The vein carrier can maintain the sterility of environment inside the translucent holder, thereby preventing contamination of the vein during the next steps of the method 600.

At block 605, the method 600 can include providing the sterile vein carrier to a light chamber. In some aspects, the closed vein carrier with the coated vein secured inside is handed out of the sterile field to a second operator. In some aspects, the second operator does not need to be scrubbed in as the vein is maintained in the sterile field within the vein carrier.

At block 606, the method 600 can include docking, mating, and/or connecting the vein carrier to the the light chamber. In some aspects, the light chamber does not need to be sterile because the vein carrier contains the vein in a sterile environment. Docking the vein carrier and the light chamber can include aligning one or more rails and/or ledges of the vein carrier and one or more rails and/or ledges of a docking bay of the light chamber. The vein carrier can then be inserted and snapped into place via a connection mechanism on the vein carrier and a connection mechanism within the light chamber's docking bay. Docking can also include electronically connecting the vein carrier and the docking bay utilizing the electrical connections described herein.

At block 607, method 600 can include closing the light chamber. In some aspects, closing the light chamber includes surrounding the vein carrier with the light chamber. In some aspects, the light chamber can be closed by moving a moveable light tower to be in contact with a fixed light tower. The moveable light tower can have a connection mechanism (e.g., magnet, magnetic material, screw/hole, latch, detent, and the like) configured to removably couple to a connection mechanism on the fixed light tower. The moveable light tower and the fixed light tower can completely enclose the vein carrier (and thereby the translucent holder) containing the vein. The operator can then start the light treatment by pressing a start button in communication with one or more light sources. In some aspects, the one or more light sources are disposed on and/or in the fixed light tower and the moveable light tower. In some aspects, the operator can select parameters of the light treatment before pressing the start button using a display (e.g., indicator screen). The parameters can include light parameters (e.g., spectrum, wavelength, intensity, etc.), temperature parameters (e.g., maximum and minimum temperatures to be maintained by fans connected to the light chamber and/or the vein carrier), humidity parameters (e.g., minimum and maximum humidity maintained in the vein carrier utilizing the fans and the humidifiers connected to the vein carrier), or other desired parameters. When the start button is pressed the light treatment begins.

At block 608, the method 600 can include removing the vein carrier from the light chamber. After the light treatment is completed, as indicated on the indicator screen and/or by indicator LEDs, the operator can remove the vein carrier from the light chamber. In some aspects, the operator can open the light chamber by moving the moveable light tower to an open configuration. The vein carrier can be removed from the light chamber by removing the vein carrier from the docking bay.

At block 609, the method 600 can include opening the vein carrier. In some aspects, opening the vein carrier includes opening the translucent holder. In some aspects, the operator (e.g., second operator) can use an upper extension tab and/or lower extension tab on the exterior of the moveable translucent section of the translucent holder to open the translucent holder. The operator (e.g., second operator) can avoid accessing the interior of translucent holder, and thereby avoid breaking sterility, by using the extension tabs and presenting the interior of the translucent holder with the treated vein to the first operator (e.g., scrubbed in operator).

At block 610, the method can include removing the vein from the vein carrier using a sterile transfer technique. The original operator (e.g., scrubbed in operator) can remove the light treated vein from the vein carrier. In some aspects, the original operator (e.g., scrubbed in operator) can reach into the inside of the translucent holder of the vein carrier and remove the treated vein in a standard sterile transfer manner. The vein carrier protects the vein from contamination during the light treatment by maintaining sterility within vein carrier (e.g., the translucent holder). Therefore, the vein has been maintained in a sterile condition during the treatment.

At block 611, the method can include implanting the vein back into the patient's body. The first operator (e.g., scrubbed in operator) can complete the by-pass anastomoses using the treated vein.

The present disclosure also relates to a vein treated with a photo-initiator and light using the systems and methods described herein. For example, the vein can have increased strength after being coated with the photo-initiator and light. The vein can be formed by a process including removing the vein from the patient, coating the vein with a photo-initiator, securing the vein within a translucent holder of a vein carrier, receiving the vein carrier in a light chamber, and providing light, via the light chamber, to the vein through the translucent holder, thereby activating the photo-initiator and modifying the vein. In some aspects, the sterility of the vein is maintained throughout the process as the vein is placed into the sterile translucent holder while in a sterile operating room. After the light treatment, the translucent holder can be presented back to the operator (e.g., surgeon who removed the vein) such that the sterility of the vein is maintained.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

### EXAMPLES

### Example 1

The systems and methods described herein were used to treat vein grafts in a first example. The purpose of this test was to characterize the tensile strength of photochemical tissue passivation (PTP) treated saphenous vein grafts and untreated saphenous vein graft control specimens.

Samples to be tested are presented in Table 1. The systems described herein were used to treat the samples. The samples (saphenous veins) were ovine sourced, which is an animal model used for evaluation the suitability of devices intended for human vascular use

TABLE 1
Test Samples

| Sample | | | Number of | |
|---|---|---|---|---|
| Description | Condition | Size | Samples | Sterilization |
| Saphenous | PTP Treated | 3-6 mm diameter | 6 | N/A |
| Vein Graft | | 6 cm length | | |
| Saphenous | Untreated | 3-6 mm diameter | 6 | N/A |
| Vein Graft | (control) | 6 cm length | | |

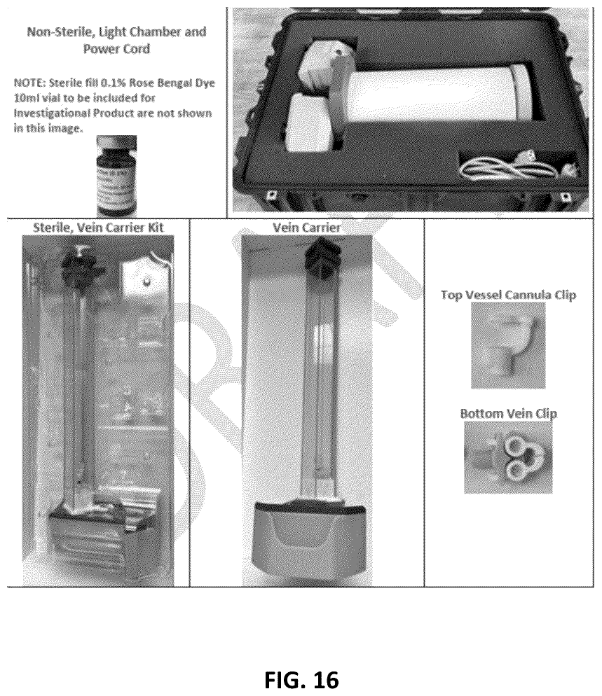

The system used in this example included the components illustrated in FIG. 16. The components of the system include the light chamber described herein, the vein carrier described herein, the vessel cannula clip described herein, the bottom vein clip described herein, and 0.1% Rose Bengal dye. The vein carrier can be packaged in a sterile vein carrier kit (e.g., sterile packaging).

The samples (saphenous vein grafts) were received at a lab. Six total samples were received. Each sample was cut into two pieces, with each piece having a length of 6 cm. One piece from each sample was PTP treated, while the other piece was not PTP treated (i.e., control), providing a total of twelve samples, as shown in Table 1.

Each sample (saphenous vein graft) was fed over a beveled end of a cannula ensuring 10-15 mm vein coverage over the cannula length. A 0-silk suture was used to secure the vein to the cannula by tying the suture around the vein approximately 5-10 mm from the tip of the cannula (e.g., approximately halfway between the cannula tip and the end of the sample (saphenous vein graft)). Overtightening the suture was avoided to prevent damage to the sample (saphenous vein graft). The suture was tied off with the minimal force required to securely attach the sample (saphenous vein graft) to the cannula.

PTP treatment was then performed on the six PTP treated samples. The light chamber was placed on a stable, level surface. The power cord was attached to the light chamber. The light chamber door was closed. The light chamber was turned on. The execution of the light chamber power up cycle was confirmed by the three LED lights illuminating in a green color.

The vein carrier was then removed from the packaging (e.g., Tyvek pouch). The fluid port plug of the vein carrier was then removed to allow access to the fluid chamber of the vein carrier. 80 mL of distilled water was added into the fluid chamber using a 50 ml syringe.

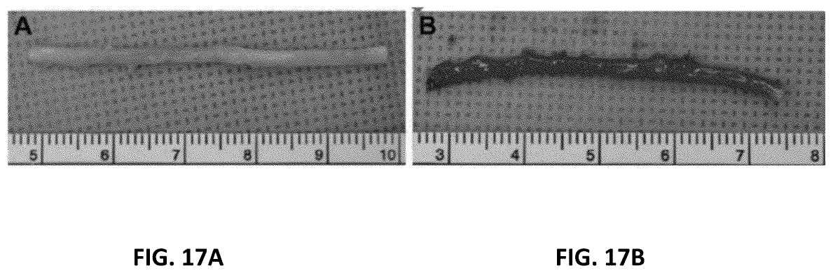

The vein carrier was then opened. A 10 mL syringe with a needle was used to draw 2 mL of Rose Bengal dye from a vial containing the Rose Bengal dye. The needle was then removed from the syringe. Using the syringe, the Rose Bengal dye was then applied to the first sample to be PTP treated. An appropriate method (e.g., brushing) using minimal pressure was used to spread the applied dye ensuring complete coverage. The first sample was then rotated, and the process was repeated to coat the other side of the first sample. FIG. 17A illustrates an uncoated sample and FIG. 17B illustrates a coated sample.

The Rose Bengal dye coated first sample was then placed in phosphate buffered saline (PBS) for five minutes. The first sample was then attached to the vein carrier using the systems and methods described herein. The vein carrier was attached to the light chamber as described herein. The first sample was then treated with light using the systems and methods described herein. The first sample was treated with light having a wavelength of about 530 nanometers to about 550 nanometers. The process was repeated for the remaining five samples to be PTP treated.

The longitudinal tensile strength was evaluated by applying a tensile force to the longitudinal axis of each sample. A universal mechanical tensile strength test system was used, where the samples were fixed to the test system. A calibrated load cell, calibrated ruler, and calibrated weights were used. A constant travel rate of 100 mm/min was applied to each individual sample, until the sample separated into two pieces. A gauge length of greater than 5 cm was used. The test environment was air. The test environment temperature was 22+/−5 degrees C. The peak load and location of failure was recorded.

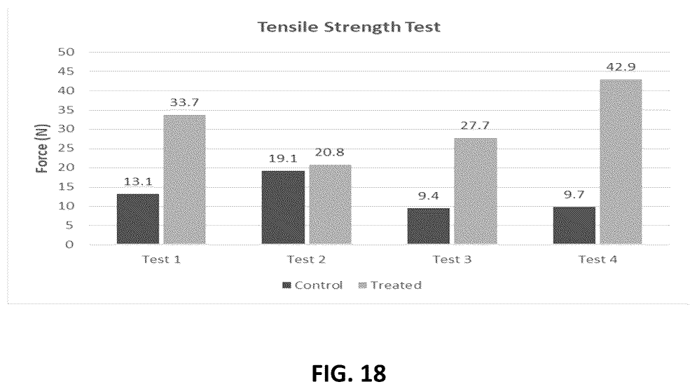

FIG. 18 illustrates the results of the test for four PTP treated samples compared to four control samples. The tensile strength of each samples was converted to a maximum force to separation. As illustrated in FIG. 18, the PTP treated samples have significantly higher maximum force to separation than the control samples. The first PTP treated sample had a maximum force to separation of 33.7 Newtons (N) compared to the first control sample with a maximum force to separation of 13.1 N. The second PTP treated sample had a maximum force to separation of 20.8 N compared to the second control sample with a maximum force to separation of 19.1 N. The third PTP treated sample had a maximum force to separation of 27.7 N compared to the second control sample with a maximum force to separation of 9.4 N. The fourth PTP treated sample had a maximum force to separation of 42.9 N compared to the fourth control sample with a maximum force to separation of 9.7 N.

### Example 2

The system and methods described herein were used to treat vein grafts in a second example. The purpose of this test was to characterize the pressurized burst strength of PTP treated saphenous vein grafts and untreated saphenous vein graft control specimens.

The samples tested are presented in Table 2. The samples used were ovine sources, which is an animal model used for evaluating the suitability of devices intended for human vascular use.

TABLE 2
Test Samples

| Sample | | | Number of | |
|---|---|---|---|---|
| Description | Condition | Size | Samples | Sterilization |
| Saphenous | PTP Treated | 3-6 mm diameter | 6 | N/A |
| Vein Graft | | 6 cm length | | |
| Saphenous | Untreated | 3-6 mm diameter | 6 | N/A |
| Vein Graft | (control) | 6 cm length | | |

The system used in this example included the components illustrated in FIG. 16. The components of the system include the light chamber described herein, the vein carrier described herein, the vessel cannula clip described herein, the bottom vein clip described herein, and 0.1% Rose Bengal dye. The vein carrier can be packaged in a sterile vein carrier kit (e.g., sterile packaging).

The samples (saphenous vein grafts) were received at a lab. Six total samples were received. Each sample was cut into two pieces, with each piece having a length of 6 cm. One piece from each sample was PTP treated, while the other piece was not PTP treated (i.e., control), providing a total of twelve samples, as shown in Table 2.

Each sample (saphenous vein graft) was fed over a beveled end of a cannula ensuring 10-15 mm vein coverage over the cannula length. A 0-silk suture was used to secure the vein to the cannula by tying the suture around the vein approximately 5-10 mm from the tip of the cannula (e.g., approximately halfway between the cannula tip and the end of the sample (saphenous vein graft)). Overtightening the suture was avoided to prevent damage the sample (saphenous vein graft). The suture was tied off with minimal force required to securely attach the sample (saphenous vein graft) to the cannula.

PTP treatment was then performed on the six PTP treated samples. The light chamber was placed on a stable, level surface. The power cord was attached to the light chamber. The light chamber door was closed. The light chamber was turned on. The execution of the light chamber power up cycle was confirmed by the three LED lights illuminating in a green color.

The vein carrier was then removed from the packaging (e.g., Tyvek pouch). The fluid port plug of the vein carrier was then removed to allow access to the fluid chamber of the vein carrier. 80 mL of distilled water was added into the fluid chamber using a 50 mL syringe.

The vein carrier was then opened. A 10 ml syringe with a needle was used to draw 2 mL of Rose Bengal dye from a vial containing the Rose Bengal dye. The needle was then removed from the syringe. Using the syringe, the Rose Bengal dye was then applied to the first sample to be PTP treated. An appropriate method (e.g., brushing) using minimal pressure was used to spread the applied dye ensuring complete coverage. The first sample was then rotated, and the process was repeated to coat the other side of the first sample. FIG. 17A illustrates an uncoated sample and FIG. 17B illustrates a coated sample.

The Rose Bengal dye coated first sample was then placed in phosphate buffered saline (PBS) for five minutes. The first sample was then attached to the vein carrier using the systems and methods described herein. The vein carrier was attached to the light chamber as described herein. The first sample was then treated with light using the systems and methods described herein. The first sample was treated with light having a wavelength of about 530 nanometers to about 550 nanometers. The process was repeated for the remaining five samples to be PTP treated. After treatment, a balloon liner was inserted into the test samples.

The pressurized burst strength was determined by applying an increasing pressure to each sample until it bursts, defined by a noticeable decline in internal pressure with an inability to maintain pressure. A calibrated pressure gauge, calibrated stop water, calibrated inflation/deflation device (e.g., pump, compressor, etc.) calibrated thermometer, calibrated hydraulic burst leak tester with pressure gauge, and calibrated ruler were used. The starting pressure was set to zero and increased using a rise rate of 70 kPa/second (525 mmHg/second). Pressure was logged using a data acquisition system, and the maximum pressure achieved before burst was recorded as the burst pressure. A balloon liner was used to prevent leakage prior to reaching the burst pressure. The test solution was distilled water at room temperature. The test sample gauge length was greater than or equal to 1 cm. The test solution temperature was 22+/−5 degrees C. The balloon liner was a compliant balloon liner with a diameter noticeably greater than the nominal pressurized diameter of the sample at 16 kPa (120 mmHg). For each sample, the maximum pressure applied, mode of failure, and location of failure was recorded.

Table 3 illustrates the results of the pressurized burst strength testing.

TABLE 3
Max Pressure Before Burst

| | | Max |
|---|---|---|
| Sample ID | Condition | Pressure (kPa) |
| PE-628-11 | Control | 113 |
| PE-628-25 | | 81 |
| PE-628-26 | | 139 |
| PE-628-31 | PTP | 438 |
| PE-628-32 | Treated | 92 |
| PE-628-33 | | 245 |
| PE-628-34 | | 168 |

As illustrated in Table 3, the maximum pressure before burst for the four PTP treated samples was significantly higher than the maximum pressure before burst of the control samples.

### Illustrative Aspects

The following is a list of non-limiting illustrative aspects and may include combinations thereof:

Aspect 1: A system for sterile tissue modification of a vein utilizing a photo-initiator and light, the system comprising: a light chamber, the light chamber comprising: a fixed light tower comprising one or more light source assemblies; a moveable light tower comprising one or more light source assemblies; and a light chamber temperature control assembly; and a vein carrier, the vein carrier comprising; a translucent holder, the translucent holder comprising: a fixed translucent section; a moveable translucent section; and a vein attachment mechanism configured to mount the vein coated in the photo-initiator within the translucent holder; and a vein carrier temperature control assembly.

Aspect 2: The system of aspect 1, wherein the moveable translucent section of the translucent holder is operable to be moved between an open configuration and a closed configuration.

Aspect 3: The system of aspect 1 or aspect 2, wherein the vein is secured to the vein carrier via the vein attachment mechanism when the moveable translucent section is in the open configuration.

Aspect 4: The system of any one of aspects 1-3, wherein the fixed translucent section is removably coupled to the moveable translucent section via an attachment mechanism when the moveable translucent section is in the closed configuration, thereby providing a sterile environment within the translucent holder.

Aspect 5: The system of any one of aspects 1-4, wherein the moveable light tower is operable to be moved between an open configuration and a closed configuration.

Aspect 6: The system of any one of aspects 1-5, the system further comprising a lock configured to lock the moveable light tower in the closed configuration.

Aspect 7: The system of any one of aspects 1-6, wherein the light chamber further comprises a docking bay having a connection mechanism operable to receive a connector of the vein carrier when the moveable light tower is in the open configuration.

Aspect 8: The system of any one of aspects 1-7, wherein the moveable light tower is moved to the closed configuration when the vein carrier is received in the docking bay.

Aspect 9: The system of any one of aspects 1-8, wherein each of the one or more light source assemblies of the fixed light tower and the moveable light tower comprises a light source and a lens configured to direct light to the translucent holder of the vein carrier.

Aspect 10: The system of aspect 9, wherein the lens has a parabolic profile to direct light to a center point of the translucent holder, thereby providing light to an entire surface area of the vein.

Aspect 11: The system of aspect 9, wherein the light source in each of the one or more light source assemblies is a green LED having a spectrum of about 530 nm to about 540 nm, or about 532 nm, a laser configured to emit a laser beam having a wavelength of about 100 nm to about 1,200 nm, a halogen bulb, a mercury vapor bulb, or combinations thereof.

Aspect 12: The system of any one of aspects 9-11, wherein the one or more light source assemblies of the fixed light tower comprises two light source assemblies.

Aspect 13: The system of any one of aspects 9-12, wherein the one or more light source assemblies of the moveable light tower comprises two light source assemblies.

Aspect 14: The system of aspect 13, wherein the two light source assemblies of the fixed light tower and the two light source assemblies of the moveable light tower provide light in a sequential order.

Aspect 15: The system of any one of aspects 1-14, wherein the light chamber temperature control assembly comprises a fan in fluid communication with one or more vents and one or more heat sinks integral to the fixed light tower and/or the moveable light tower.

Aspect 16: The system of aspect 15, wherein the light chamber temperature control assembly further comprises a second fan in fluid communication with the one or more vents, the second fan operable to draw air out of the light chamber temperature control assembly.

Aspect 17: The system of any one of aspects 1-16, wherein the vein carrier temperature control assembly comprises a fan and a cooling vent in fluid communication with the translucent holder.

Aspect 18: The system of aspect 17, wherein the vein carrier temperature control assembly further comprises one or more humidifiers and a fluid chamber in fluid communication with the fan and the cooling vent operable to provide moisture to the translucent holder.

Aspect 19: The system of aspect 18, wherein the vein carrier temperature control assembly further comprises a second fan in fluid communication with an air outtake vent, the second fan configured to draw air out of the translucent holder.

Aspect 20: The system of aspect 19, wherein the fluid chamber contains a fluid comprising saline, water, or lactated ringers' solution.

Aspect 21: The system of any one of aspects 1-20, wherein the vein attachment mechanism has an adjustable length, and the vein is secured to the vein attachment mechanism in a vertical position or a “U” shaped position.

Aspect 22: The system of aspect 21, wherein the vein has a first end and a second end defining a vein length.

Aspect 23: The system of aspect 22, wherein the vein length is about 5 cm to about 40 cm.

Aspect 24: The system of aspect 22 or aspect 23, wherein the vein attachment mechanism comprises: a vessel cannula configured to secure the first end of the vein; a vessel cannula clip configured to removably couple to the vessel cannula, the vessel cannula clip comprising a vessel cannula clip magnetic material; an upper magnetic holder located on an upper interior surface of the translucent holder, the upper magnetic holder having an upper magnetic holder magnet configured to removably couple to the vessel cannula clip magnetic material; a lower vein clamp configured to secure the second end of the vein, the lower vein clamp further comprising a lower vein clamp magnetic material; a guide rail spanning the translucent holder from the upper interior surface of the translucent holder to a bottom interior surface of the translucent holder; and a floating bracket moveably coupled to the guide rail, the floating bracket comprising a lower magnetic holder having a lower magnetic holder magnet configured to removably couple to the lower vein clamp magnetic material, wherein the floating bracket allows a length of the vein attachment mechanism to be adjusted.

Aspect 25: The system of any one of aspects 22-24, wherein the vein attachment mechanism comprises: a vessel cannula configured to secure the first end of the vein; a vessel cannula clip configured to removably couple to the vessel cannula, the vessel cannula clip comprising a vessel cannula clip magnetic material; a vein clamp configured to secure the second end of the vein, the vein clamp comprising a vein clamp magnetic material; an upper magnetic holder located on an upper interior surface of the translucent holder, the upper magnetic holder comprising an upper magnetic holder magnet configured to removably couple to the vessel cannula clip magnetic material and the vein clamp magnetic material; a guide rail spanning the translucent holder from the upper interior surface of the translucent holder to a lower interior surface of the translucent holder; and a floating bracket moveably coupled to the guide rail, the floating bracket comprising a semi-circular holder having one or more arms, wherein the one or more arms are configured to secure a portion of the vein between the first end and the second end of the vein, wherein the floating bracket allows a length of the vein attachment mechanism to be adjusted.

Aspect 26: The system of any one of aspects 1-25, wherein the system comprises one or more sensors comprising a temperature sensor, a humidity sensor, a light sensor, or combinations thereof.

Aspect 27: The system of aspect 26, wherein the system comprises a control system, the control system configured to: turn the light chamber on to provide a light treatment to the vein; receive data from the one or more sensors; control a temperature and humidity of the system via the light chamber temperature control assembly and the vein carrier temperature control assembly based on the data from the one or more sensors; and cause a display to display information related to the light treatment.

Aspect 28: The system of aspect 27, wherein the control system is operable to turn off the one or more light source assemblies when the moveable light tower is in an open configuration.

Aspect 29: The system of aspect 27 or aspect 28, wherein the control system is configured to keep the light chamber temperature control assembly and the vein carrier temperature control assembly on the entire time the vein carrier is docked within the light chamber.

Aspect 30: The system of any one of aspects 27-29, wherein the display is configured to display one or more warnings or errors.

Aspect 31: The system of aspect 30, wherein the one or more warnings or errors comprise one or more of temperature warnings, humidity warnings, light source warnings, and connection warnings.

Aspect 32: The system of any one of aspects 27-31, wherein the control system is in electronic communication with a modem configured to transmit data to a cloud network to ensure correct and safe operable of the system.

Aspect 33: A method for modifying a tissue, the method comprising: obtaining the tissue from a patient; coating the tissue with a photo-initiator; securing the tissue in a translucent holder of a vein carrier; and providing light to the translucent holder of the vein carrier via a light chamber to modify the tissue.

Aspect 34: The method of aspect 33, wherein the photo-initiator is xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, Rose Bengal (“RB”), erythrosine, riboflavin, methylene blue (MB), Toluidine Blue, Methyl Red, Janus Green B, Rhodamine B base, Nile Blue A, Nile Red, Celestine Blue, Remazol Brilliant Blue R, riboflavin-5-phosphate (R-5-P), N-hydroxypyri-dine-2-(I H)-thione (N-HTP), or photoactive derivatives thereof.

Aspect 35: The method of aspect 33 or aspect 34, wherein the tissue is maintained in a sterile condition within the translucent holder.

Aspect 36: The method of any one of aspects 33-35, the method further comprising implanting the modified tissue into the patient.

Aspect 37: The method of any one of aspects 33-36, wherein the light is provided by one or more light sources comprising a green LED having a wavelength of about 530 nm to about 540 nm, or about 532 nm, a laser configured to emit a laser beam having a wavelength of about 100 nm to about 1,200 nm, a halogen bulb, a mercury vapor bulb, or combinations thereof.

Aspect 38: The method of aspect 37, wherein the one or more light sources are four light sources configured to provide light sequentially for about 5 seconds to about 15 seconds, or about 10 seconds.

Aspect 39: The method of aspect 38, wherein two or more of the four light sources provide light simultaneously.

Aspect 40: A modified vein formed using any one of the systems of Aspects 1-32.

Aspect 41: A modified vein formed by any of one of the methods of Aspects 33-39.

What is claimed:

1. A system for tissue modification of a vein, the system comprising:
a vein carrier comprising:
a translucent holder; and
a vein attachment mechanism disposed within the translucent holder; and
a light chamber configured to receive the vein carrier, the light chamber comprising one or more light assemblies configured to provide light to the translucent holder of the vein carrier.

2. The system of claim 1, wherein the light chamber comprises a fixed light tower and a moveable light tower, wherein the fixed light tower and the moveable light tower are operable to surround the vein carrier.

3. The system of claim 1, wherein the translucent holder comprises a fixed translucent section and a moveable translucent section.

4. The system of claim 1, wherein the light chamber further comprises a docking bay having a connection mechanism operable to receive a connector of the vein carrier.

5. The system of claim 1, wherein the translucent holder is operable to maintain sterility of the vein.

6. The system of claim 1, wherein each of the one or more light assemblies comprises at least one light source and at least one lens configured to direct light to the vein through the translucent holder.

7. The system of claim 1, wherein the vein carrier further comprises a vein carrier temperature control assembly operable to cool and/or humidify the vein within the translucent holder.

8. The system of claim 7, wherein the vein carrier temperature control assembly includes at least one fan, at least one vent, a fluid chamber, and one or more humidifiers.

9. The system of claim 1, wherein the light chamber further comprises a light chamber temperature control assembly operable to cool the light chamber.

10. The system of claim 9, wherein the light chamber temperature control assembly includes at least one fan, one or more vents, and one or more heat sinks coupled to the one or more light assemblies.

11. The system of claim 1, wherein the vein attachment mechanism has an adjustable length.

12. The system of claim 1, wherein the system further comprises a controller operable to control one or more parameters of the system.

13. The system of claim 12, wherein the one or more parameters include temperature, humidity, light intensity, light frequency, light wavelength, and/or light spectrum.

14. A system for tissue modification of a vein, the system comprising:
a light chamber comprising:
a fixed light tower comprising one or more light source assemblies;
a moveable light tower comprising one or more light source assemblies; and
a light chamber temperature control assembly; and
a vein carrier operable to be received within the light chamber, the vein carrier comprising:
a translucent holder operable to secure the vein, the translucent holder comprising:
a fixed translucent section;
a moveable translucent section; and
a vein attachment mechanism operable to secure the vein within the translucent holder; and
a vein carrier temperature control assembly operable to control a temperature and/or humidity within the translucent holder,
wherein the translucent holder is configured to maintain sterility of the vein.

15. A method for modifying a vein, the method comprising:
obtaining the vein from a patient;
coating the vein with a photo-initiator;
securing the vein within a translucent holder of a vein carrier, the translucent holder operable to maintain sterility of the vein;
receiving the vein carrier in a light chamber; and
providing a light, via the light chamber, to the vein through the translucent holder, thereby activating the photo-initiator and modifying the vein.

16. The method of claim 15, wherein the light comprises a wavelength of about 530 nm to about 540 nm.

17. The method of claim 15, wherein the photo-initiator includes one or more of xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, Rose Bengal (“RB”), erythrosine, riboflavin, methylene blue (MB), Toluidine Blue, Methyl Red, Janus Green B, Rhodamine B base, Nile Blue A, Nile Red, Celestine Blue, Remazol Brilliant Blue R, riboflavin-5-phosphate (R-5-P), N-hydroxypyri-dine-2-(I H)-thione (N-HTP), or photoactive derivatives thereof.

18. The method of claim 15, wherein the light is provided by two or more light sources, wherein the two or more light sources provide the light sequentially.

19. The method of claim 15, the method further comprising implanting the vein into the patient.

20. The method of claim 15, the method further comprising maintaining a temperature and/or humidity within the translucent holder via a vein carrier temperature control assembly.
